SEQUENCE LISTING (7 sequences)

SEQ ID NO: 1 - protein, 370 aa, Homo sapiens <s300> NCBI/SWISS PROT Accession No. P04628 2005-05-01 (1)..(370)
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 2 - protein, 21 aa, Homo sapiens
QNPGILHSVSGGLQSAVRECK

SEQ ID NO: 3 - protein, 14 aa, Homo sapiens
WNCPTAPGPHLFGK

SEQ ID NO: 4 - protein, 20 aa, Homo sapiens
GPGGPDWHWGGCSDNIDFGR

SEQ ID NO: 5 - protein, 20 aa, Homo sapiens
FLMNLHNNEAGRTTVFSEMR

SEQ ID NO: 6 - protein, 19 aa, Homo sapiens
SPNFCTYSGRLGTAGTAGR

SEQ ID NO: 7 - protein, 12 aa, Homo sapiens
NCTHTRVLHECL

(19) United States
(12) Patent Application Publication
(10) Pub. No.: US 2007/0117100 A1
(43) Pub. Date: May 24, 2007

(54) Biomarker for determining predisposition and/or prognosis of hepatocellular carcinoma

(76) Inventor: Sun-Lung Tsai, Yung Kang City (TW)

Correspondence Address: Paul D. Greeley, Esq.;Ohlandt, Greeley, Ruggiero & Perle, L.L.P., 10th Floor, One Landmark Square, Stamford, CT, 06901-2682, US

(21) Appl. No.: 11/282,888

(22) Filed: Nov. 18, 2005

(51) Int. Cl. C12Q 1/68; G01N 33/574

(52) U.S. Cl. 435/6; 435/7.23

(57) ABSTRACT

Disclosed herein are a method for determining predisposition to hepatocellular carcinoma (HCC) in a subject having or suspected to have a hepatic nodule, in particular one infected with at least one of hepatitis B virus and hepatitis C virus, and a method for prognostic evaluation of a subject having or suspected to have HCC, in which Wnt-1 is used as a biomarker of HCC.

### BACKGROUND OF THE INVENTION

1. Field of the Invention

The present invention relates to the finding of Wnt-1 as a biomarker of hepatocellular carcinoma (HCC). Based on said finding, the present invention provides a method for determining predisposition to HCC in a subject having or suspected to have a hepatic nodule, in particular one infected with at least one of hepatitis B virus and hepatitis C virus, and a method for prognostic evaluation of a subject having or suspected to have HCC, in which Wnt-1 is used as a biomarker of HCC.

2. Description of the Related Art

One of the most important factors in the survival from cancer is detection at an early stage. Clinical assays that detect the early events of cancer offer an opportunity to intervene and prevent cancer progression. With the development of gene profiling and proteomics, there has been significant progress in the identification of molecular markers or “biomarkers” that can be used to diagnose and prognose specific cancers.

For example, U.S. Pat. No. 5,866,323 issued to Sanford D. Markowitz et al. discloses a method for diagnosis or prognosis of cancer by detection of the absence of functional type II receptor for TGF-β(RII) in cells of a patient.

U.S. Pat. No. 6,303,324 B1 issued to John Fruehauf et al. discloses a method for making a prognosis of disease course in a human cancer patient, the method comprising the steps of: (a) obtaining a sample of a tumor from the human cancer patient; (b) determining a level of nuclear localization of p53 protein in the tumor sample and comparing the level of nuclear localization of p53 protein in the tumor sample and comparing the level of nuclear localization of p53 protein in the tumor sample with the level of nuclear localization of p53 protein in a non-invasive, non-metastatic tumor sample; (c) determining a level of thrombospondin 1 expression in the tumor sample and comparing the level of thrombospondin 1 expression in a non-invasive, non-metastatic tumor sample; (d) determining by immunohistochemistry an extent of microvascularization in the tumor sample and comparing the extent of microvascularization in the tumor sample with the extent of microvascularization in a non-invasive, non-metastatic tumor sample; wherein said prognosis is predicted from considering a likelihood of further neoplastic disease which is made when the level of nuclear localization of in the tumor sample is greater than the level of nuclear localization of p53 protein in the non-invasive, non-metastatic tumor sample; the level of thrombospondin 1 expression in the tumor sample is less than the level of thrombospondin 1 expression in the non-invasive, non-metastatic tumor sample; and the extent of microvascularization in the tumor sample is greater than the extent of microvascularization in the non-invasive, non-metastatic tumor sample; and wherein the human cancer patient has breast cancer or prostate cancer.

U.S. Pat. No. 6,607,894 B1 issued to Alexander Lopata et al. discloses methods for assaying the presence and/or risk of endometrial cancer by measurement of levels of matrix metalloproteinase-2 and/or matrix metalloproteinase-9 in uterine washings. The methods may be qualitative or quantitative, and are adaptable to large-scale screening and to clinical trials.

US patent application Publication No 20050048542 A1 discloses a non-invasive, quantitative test for prognosis determination in cancer patients. The test relies on measurements of the tumor levels of certain messenger RNAs (mRNAs) or the corresponding gene expression products. These mRNA or protein levels are entered into a polynomial formula (algorithm) that yields a numerical score, which indicates recurrence risk (recurrence score) or the likelihood of patient response to therapy (response score).

WO 2005/071387 A1 discloses methods for the diagnosis and prognosis of cancers of epithelial origin by assessing levels of ADAM 12 in a biological sample obtained from a patient.

Even though there has been significant progress in the field of cancer detection, there still remains a need in the art for the identification of new biomarkers for a variety of cancers that can be easily used in clinical applications.

Hepatitis B virus (HBV) and hepatitis C virus (HCV) infect more than 350 and 170 million people worldwide, respectively (Purcell, R. H. (1993), Gastroenterology, 104:955-963). Both viruses share common features in chronically-infected subjects, including similar histopathological changes in the liver, and common clinical evolution from chronic hepatitis, liver cirrhosis and ultimately to hepatocellular carcinoma (HCC)(S. L. Tsai and Y. F. Liaw (1995), Digest. Surg., 12:7-15; K. Okuda (1992), Hepatology, 15:948-963; S. S. Thorgeirsson and J. W. Grisham (2002), Nature Genet., 31:339-346).

For patients with chronic viral hepatitis, screening for early-stage HCC may permit the institution of curative treatment strategies, and antiviral treatment may reduce the risk of subsequent development of HCC. For patients with established hepatocellular carcinoma, the presence of concurrent chronic viral hepatitis or cirrhosis may affect prognosis and survival and may alter treatment options because of impaired hepatic function.

Despite recent advances in diagnostic methods for HCC, the prognosis is still generally poor. Patients with metastatic or locally advanced HCC usually respond poorly to anticancer treatments. While untreated patients usually die in 3-4 months, treated patients may live 6 to 18 months if they respond to therapy. Long-term survival is seen occasionally after successful subtotal hepatectomy for non-invasive carcinoma. Because the normal metabolic and storage functions of the liver are impaired, patients are at risk for nutritional and bleeding complications. Patients with advanced cirrhosis commonly succumb to complications, such as encephalopathy, variceal hemorrhage, and sepsis, independently of the tumor's extent.

Hepatic resection remains the mainstay of treatment of this tumor and provides the only consistent long-term survival (N. Nagasue et al. (2001), British Journal of Surgery, 88:515-522). At present, hepatic resection is only feasible for 10-15% of patients. The reasons for this low resectability rate include extensive local disease, presence of extrahepatic disease and poor functional liver reserve precluding any form of hepatic resection (T. K. Seow et al. (2001), Proteomics, 1:1249-1263).

As HCC can reach an advanced stage before it presents clinically, regular screening at six monthly intervals is recommended for those at risk individuals. This entails the performance of a transabdominal ultrasound scan of the liver to detect for the presence of tumor nodule(s) and the measurement of the serum tumor marker α-feto-protein (AFP)(T. K. Seow et al. (2001), Proteomics, 1:1249-1263).

Several studies have reported proteomic analysis of HCC, either from tumor tissues (J. Kim et al. (2002), Electrophoresis. 23: 4142-4156; S. O. Lim et al. (2002), Biochem. Biophys. Res. Commun., 291: 1031-1037; K. S. Park et al. (2002), Int. J, Cancer, 97: 261-265), or from patients' sera (F. L. Naour et al. (2002), Mole. Cell. Proteomics., 1:197-203). While all attempted to identify specific factors associated with hepatocarcinogenesis or novel tumor markers for early diagnosis of HCC, these goals seem far from reach (R. C. M. Y. Liang et al. (2002), J. Chromatogr. B., 771:303-328; T. K. Seow et al. (2001), Proteomics, 1:1249-1263).

While previously identified markers, such as AFP, serum ferritin, γ-glutamyltranspeptidase isoenzyme, alkaline phosphatase, des-γ-carboxy prothrombin, α-1-antitrypsin, aldolase A, 5′-nucleotide phosphodiesterase, tissue polypeptide antigen, and α-1-fucosidase (T. K Seow et al. (2001), Proteomics, 1; 1249-1263) have facilitated efforts to diagnose and treat HCC, there is still a need for the identification of additional markers and therapeutic targets for HCC in order to improve further the diagnosis and therapy of this tumor.

Wnt genes encode a family of 38-45 kDa, secreted cysteine-rich proteins lacking transmembrane domains that are modified by N-linked glycosylation (K. M. Cardigan and R. Nusse (1997), Genes Dev., 11:3286-3305). These secreted Wnt proteins associate with extracellular matrix proteins on or near the cell surface and, can exert autocrine or paracrine effects. The first member of the 19 known human Wnt genes, Wnt-1, was first discovered because of its oncogenic properties (R. Nusse and H. E. Varmus (1982), Cell, 31:99-109). The subsequent discovery of wingless, the fly homolog of Wnt-1, paved the way for assembling a signaling pathway found to contain cancer-causing genes (K. M. Cardigan and R. Nusse (1997), Genes Dev., 11:3286-3305; P. Polakis (2000), Genes Dev., 14:1837-1641).

There are numerous reports on the overexpression, and sometimes underexpression of Wnt genes in human cancers (K. M. Cardigan and R. Nusse (1997), supra; P. Polakis (2000), supra; J. Taipale, and P. A. Beachy (2001), Nature, 411: 349-354; D. Kalderon (2002), Trends Cell. Biol., 12: 523-531; Ariel Ruiz i Altaba et al. (2002), Nature Rev. Cancer, 2 (5): 361-370; J. R. Miller, et al. (1999), Oncogene. 18:7860-7872), and on dysregulated Wnt signaling in hematological malignancies (F. J. T. Staal and H. C. Clevers (2005), Nature Rev. Immunol., 5: 21-30). More compelling evidence directed to the amplification, rearrangement or mutation of genes encoding Wnt ligands or receptors can be found in, e.g., P. Polakis (2000), supra; J. Taipale, and P. A. Beachy (2001), supra; D. Kalderon (2002), supra; Ariel Ruiz i Altaba et al. (2002), supra; J. R. Miller, et al. (1999), supra), in which Wnt mutations were reported to occur in 85% of colorectal cancer, 74% of desmoid tumor, and 67% of hepatoblastoma.

Nuclear factor κB (NF-κB) is an important transcription factor that regulates many inflammatory and immunologic proteins, such as cytokines, interferons, major histocompatibility complex proteins, adhesion molecules, and inducible nitric oxide synthetase. Consequently, NF-κB plays an important role in cell physiology and control of apoptosis. NF-κB is a dimer of Rel proteins and usually consists of two sub-units, RelA (p65) and NFκB1 (p50). Under resting conditions, the NF-κB dimer is sequestered in the cytoplasm through interaction with an inhibitory κB (IκB) protein that prevents the NF-κB dimer from entering the nucleus. When the cell is activated by a stimulus, IκB protein is phosphorylated and degrades rapidly. The free-form NF-κB will be translocated into the nucleus and binds with the intronic enhancer of target genes to induce gene transcriptions.

Accumulating evidence indicates that both HBV (M. Doria et al. (1995), EMBO J., 14:4747-57; F. Su and R. J Schneider (1996), J. Virol., 70: 4558-4566; R. Weil et al. (1999), Mol. Cell Biol., 19: 6345-6354; J. Diao et al. (2001), Cytokine Growth F. R., 12:189-205; H. Kim et al. (2001), Biochem. Biophys. Res. Commun., 286:886-894) and HCV (D. I. Tai et al. (2000), Hepatology, 31: 656-664; H. Yoshida et al. (2001), J. Biol. Chem., 276: 16399-16405; P. Boya et al. (2001), Hepatology, 34: 1041-48; G. Gong et al. (2001), Proc. Natl. Acad. Sci. USA, 98:9599-9604; H. Marusawa et al. (1999), J. Virol., 73: 4713-4720) may activate nuclear factor-kappa B (NF-κB). Constitutive and/or inducible activation of NF-κB has been established in HBV-positive cell line Hep3B and HCV-transfected HepG2 cells, as well as in HBV- and HCV-infected liver tissues (D. I. Tai et al. (2000), Hepatology, 31: 656-664; P. J. Chiao et al. (2002), Cancer, 95:1696-1705; 0.1. Tai et al. (2000), Cancer, 89: 2274-2281). The activated NF-κB could be demonstrated by immunohistochemical staining, electrophoretic mobility shift assay (EMSA), and supershift assay. The importance of NF-κB in immunity is undisputed (W. C. Sha (1998), J. Exp. Med., 187:143-146; Q. Li (2002), Nature Rev. Immunol., 2:725-734). Recent evidence indicates that NF-κB and its activation pathways are also important for tumor development (D. Hanahan and R. A. Weinberg (2000), Cell, 100:57-70; M. Karin et al. (2002), Nature Rev. Cancer, 2: 301-310; E. Pikarsky et al. (2004), Nature, 431: 461-466; A. Lin and M. Karin (2003), Semin. Cancer Biol., 13: 107-114).

Based on previous reports in connection with NF-κB activation, it is proposed that there exist possible mechanisms of NF-κB-related hepatocarcinogenesis common to both HBV and HCV. To test this hypothesis, we analyzed NF-κB activation in paired tumor and non-tumor tissues taken from HBV- and/or HCV-infected patients, respectively. Surprisingly, we found that higher levels of NF-κB-associated Wnt-1 protein were detected in tumor portions than in non-tumor portions of paired liver specimens taken from patients infected with at least one of HBV and HCV. In addition, the enhanced expression of Wnt-1 is clinically relevant to the development of HCC in patients infected with at least one of HBV and HCV.

To our knowledge, it has not been reported in literature that Wnt-1 is closely related to the development of HCC. Based on our findings, it is possible to develop a method for determining predisposition to HCC in a subject having or suspected to have a hepatic nodule, in particular one infected with at least one of HBV and HCV, and a method for prognostic evaluation of a subject having or suspected to have HCC, in which Wnt-1 is used as a biomarker of HCC.

### SUMMARY OF THE INVENTION

Therefore, according to a first aspect, this invention provides a method for determining predisposition to hepatocellular carcinoma in a subject having or suspected to have a hepatic nodule, comprising:
separating a liver specimen taken from the subject into a tumor portion and a non-tumor portion;
detecting the levels of Wnt-1 expression in the tumor portion and the non-tumor portion, respectively;
comparing the detected level of Wnt-1 expression in the tumor portion with that in the non-tumor portion to obtain a value of ratio; and
determining whether or not the subject is predisposed to hepatocellular carcinoma based on the obtained value of ratio, wherein the subject is determined to be predisposed to hepatocellular carcinoma if the obtained value of ratio is greater than 1.

In a second aspect, this invention provides a method for prognostic evaluation of a subject having or suspected to have hepatocellular carcinoma, comprising:
separating a liver specimen taken from the subject into a tumor portion and a non-tumor portion;
detecting the levels of Wnt-1 expression in the tumor portion and the non-tumor portion, respectively;
comparing the detected level of Wnt-1 expression in the tumor portion with that in the non-tumor portion to obtain a value of ratio; and
evaluating the prognosis of the subject based on the obtained value of ratio, wherein the subject is evaluated to have:
(i) a prognosis of no more than 6 months if the obtained value of ratio is greater than 2;
(ii) a prognosis of 6 to 18 months if the obtained value of ratio is between 1 and 2; or
(iii) a prognosis of at least 18 months if the obtained value of ratio is less than 1.

### BRIEF DESCRIPTION OF THE DRAWINGS

The above and other features and advantages of the present invention will become apparent in the following detailed description of the preferred embodiments with reference to the accompanying drawing, of which:

FIG. 1 schematically shows the study protocols of this invention, in which proteins extracted from liver tissues of each paired-HCC specimens that were separated into tumor and non-tumor portions were subjected to various experiments; abbreviations: EMSA, electrophoretic mobility shift assay; IP, immunoprecipitation; 2-DE, two-dimensional polyacrylamide gel electrophoresis; and MALDI-Q-TOF, matrix-assisted laser desorption/ionization-quadrupole-time-of-flight;

FIG. 2 shows the EMSA and supershift assay results of extracted nuclear proteins as probed with anti-NF-κB p50 antibody (anti-p50), the extracted nuclear proteins being respectively obtained from the tumor and non-tumor portions of the specimens of HCC patient nos. 1-7 listed in Table 1, infra, in which abbreviations: T, nuclear proteins extracted from the tumor portions of HCC specimens; and NT, nuclear proteins extracted from the non-tumor portions of HCC specimens;

FIG. 3 shows the EMSA results of extracted nuclear proteins that were respectively obtained from the tumor and non-tumor portions of the specimens of HCC patient nos. 8-9 listed in Table 1, infra, in which abbreviations: T, nuclear proteins extracted from the tumor portions of HCC specimens; and NT, nuclear proteins extracted from the non-tumor portions of HCC specimens;

FIG. 4 shows the silver staining results of a 2-DE gel, in which proteins (500 μg) extracted from the tumor portions of the specimens of the nine HCC patients listed in Table 1, infra, were respectively subjected to immunoprecipitation (IP) using anti-p50, and the resultant immunocomplexes in the protein samples of the 9 HCC patients were collected and pooled together to run a two-dimensional electrophoresis (2-DE) analysis using a 12.5% polyacrylamide gel, followed by silver staining the thus-obtained 2-DE gel, on which the position of spot M1205434 was marked;

FIG. 5 shows the silver staining results of a 2-DE gel, in which proteins (500 μg) extracted from the tumor portions of the specimens of the nine HCC patients listed in Table 1, infra, were respectively subjected to immunoprecipitation (IP) using anti-NF-κB p65 antibody (anti-p65), and the resultant immunocomplexes in the protein samples of the 9 HCC patients were collected and pooled together to run a 2-DE analysis using a 12.5% polyacrylamide gel, followed by silver staining the thus-obtained 2-DE gel, on which the position of spot M1205434 was marked;

FIG. 6 shows the silver staining results of a 2-DE gel, in which pooled total proteins from the tumor portions of the specimens of the nine HCC patients listed in Table 1, infra, were subjected to a 2-DE analysis using a 12.5% polyacrylamide gel, followed by silver staining the thus-obtained 2-DE gel, on which the positions of 20 spots that were equivalent to those positively stained on the 2-DE gels processed with IP using anti-p50 and/or anti-p65 were arbitrarily selected and mapped together using a Typhoon 9200 ImageMaster (Amersham Biosciences) in combination with the ImageMaster 2D Platinum Software, version 5.0 (Amersham Biosciences) (Hubbard, M. J., and McHugh, N. J. (2000), Electrophoresis, 21:3785-3796);

FIG. 7 shows the mass spectrometric analysis results of 20 spots obtained from a SYPRO-Ruby-stained 2-DE gel run under conditions identical to those used for the silver-stained 2-DE gel of FIG. 6 and corresponding to those spots indicated in FIG. 6;

FIGS. 8-10 respectively show the volume comparison results of spot MI205434 as identified in FIG. 6 between the tumor and non-tumor portions of the specimens of HCC patient nos. 1, 5 and 9 listed in Table 1, infra, in which the volume comparison of spot M1205434 between the tumor and non-tumor liver tissues was made by ImageMaster (Amersham Biosciences), and the reference value was the volume obtained from the corresponding spot in the non-tumor liver tissue, which acts as a baseline value for comparison;

FIG. 11 shows the 2-DE Western blot results of the tumor and non-tumor portions of the specimens of HCC patient nos. 1 and 5 listed in Table 1, infra, in which proteins extracted from the tumor and non-tumor portions of the specimens of HCC patient nos. 1 and 5 listed in Table 1 were respectively subjected to 2-DE, followed by Western-blotting probed with anti-human Wnt-1 antibodies; abbreviations: T, HCC tumor portion; and N, HCC non-tumor portion;

FIG. 12 shows the densitometry analysis results on one-dimensional electrophoresis (1-DE)-Western blots of the tumor and non-tumor portions of additional eight-paired HCC specimens (patient nos. 10-17) acquired from the Tumor and Serum Bank of Chi-Mei Medical Center, in which proteins extracted from the tumor and non-tumor portions of the additional eight-paired HCC specimens were subjected to 1-DE analysis using a 12.5% polyacrylamide gel, followed by Western-blotting probed with anti-human Wnt-1 antibodies, and the expression level of Wnt-1 protein in each of the tumor and non-tumor portions of the additional eight-paired HCC specimens was determined by semi-quantitative analysis using the ImageMaster (Amersham Biosciences); the reference value 100 was the total counts obtained from the Wnt-1 protein band of the tumor tissue of Patient 17, which acts as a baseline value for comparison; Patients 10-13 were HCV-infected subjects, whereas Patients 14-17 were HBV-infected subjects; abbreviations: T, tumor tissue; NT, non-tumor tissue; and the arrow indicates the position of Wnt-1; and

FIG. 13 shows a postulated pathway of the development of HCC, in which activation of the Wnt-1 protein via the NF-κB signaling route bears a causal relationship to the hepatitis B- and hepatitis C-related hepatocarcinogenesis.

### DETAILED DESCRIPTION OF THE INVENTION

For the purpose of this specification, it will be clearly understood that the word “comprising” means “including but not limited to,” and that the word “comprises” has a corresponding meaning.

It is to be understood that, if any prior art publication is referred to herein, such reference does not constitute an admission that the publication forms a part of the common general knowledge in the art, in Taiwan or any other country,

Unless defined otherwise, all technical and scientific terms used herein have the meaning commonly understood by a person skilled in the art to which this invention belongs.

Various parenchymal liver diseases may lead to hepatitis, fibrosis, and eventually cirrhosis. Cirrhotic liver contains regenerative nodules and may also contain dysplastic nodules as well as hepatocellular carcinoma (HCC). Since 1995, a modified nomenclature has categorized hepatic nodules into two groups: regenerative lesions and dysplastic or neoplastic lesions.

Dysplastic or neoplastic lesions are composed of hepatocytes that show histologic characteristics of abnormal growth caused by a presumed or proved genetic alteration. Dysplastic or neoplastic nodules include hepatocellular adenoma, dysplastic foci, dysplastic nodules, and HCC.

A dysplastic focus is defined as a cluster of hepatocytes less than 1 mm in diameter with dysplasia but without definite histologic criteria for malignancy. Dysplasia indicates the presence of nuclear and cytoplasmic changes, such as minimal to severe nuclear atypia and an increased amount of cytoplasmic fat or glycogen, within the cluster of cells that compose the focus. Dysplastic foci are common in cirrhosis and uncommon in noncirrhotic livers. The dysplasia can be of the small or large cell type.

A dysplastic nodule is a nodular region of hepatocytes at least 1 mm in diameter with dysplasia but without definite histologic criteria for malignancy. These nodules are usually found in cirrhotic livers. Dysplastic nodules can be low grade or high grade (7). Nodules with low-grade dysplasia may show an altered liver parenchymal structure as well as an increased number of cells with an increased nuclei-to-cytoplasm ratio. Nodules with high-grade dysplasia show increased thickness of the layers of hepatocytes, which contain nuclei that are variable in size and shape.

HCC is a malignant neoplasm composed of cells with hepatocellular differentiation. A small HCC is defined as less than or equal to 2 cm in diameter. The criteria used to distinguish HCC from high-grade dysplastic nodules are not clearly defined. Most small HCCs cannot be distinguished histologically from dysplastic nodules with certainty. In addition, foci of carcinoma can be found in otherwise benign dysplastic nodules.

The above descriptions are excerpted from Shahid M. Hussain et al. (2002), RadioGraphics, 22:1023-1039, the disclosure of which is incorporated herein by reference in its entirety.

Chronic infections with HBV and HCV are etiologically linked to hepatitis, liver cirrhosis, and hepatocellular carcinoma (HCC). Both viruses have been reported to induce NF-κB activation in hepatocytes. In order to explore the possible mechanism(s) of NF-κB-related hepatocarcinogenesis common to both HBV and HCV, we analyzed NF-κB associated protein complexes in paired tumor and non-tumor liver tissues taken from patients infected with at least one of HBV and HCV. The quantity of NF-κB-associated proteins was semi-quantitatively measured by protein spot intensity on 2-DE gels. Protein spots associated with NF-κB signaling complexes were studied by functional proteomics analysis (R. Aebersold and M. Mann (2003), Nature, 422:198-207; E. Phizicky et al. (2003), Nature, 422:208-215).

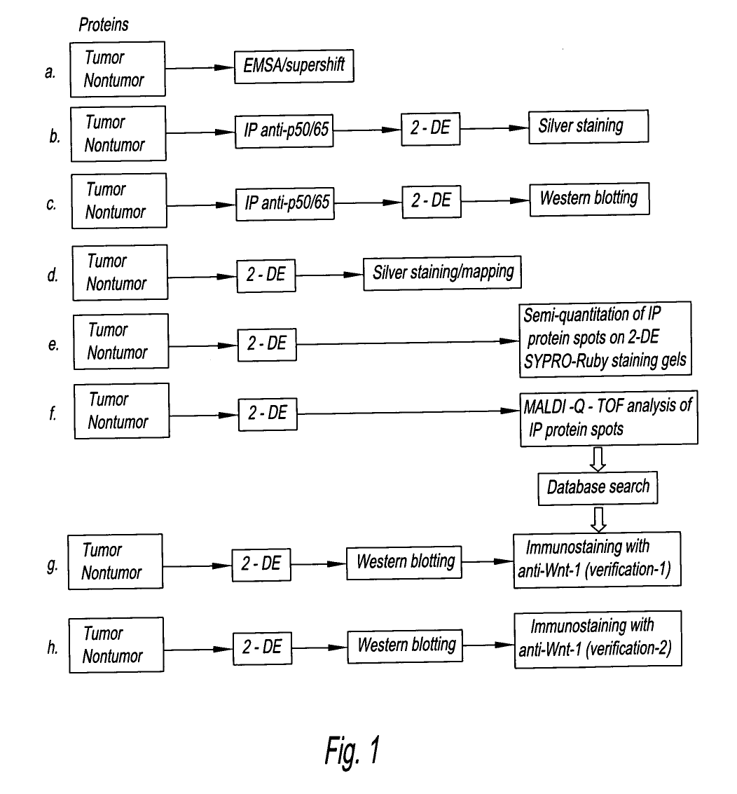

The study protocols of this invention are schematically shown in FIG. 1. Briefly, paired tumor/non-tumor liver tissues taken from patients infected with at least one of HBV and HCV were subjected to the following analyses: EMSA/supershift assay, immunoprecipitation (IP), two-dimensional polyacrylamide gel electrophoresis (2-DE), silver staining and/or SYPRO-Ruby staining, MALDI-Q-TOF analysis of IP protein spots, and Western blotting using anti-Wnt-1 antibodies.

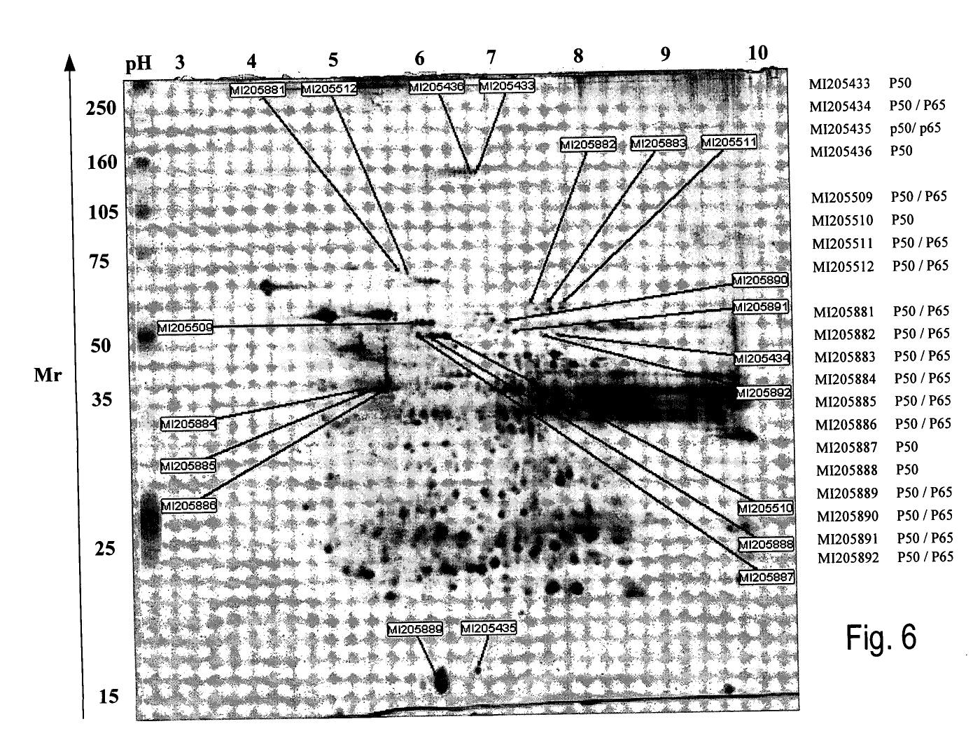

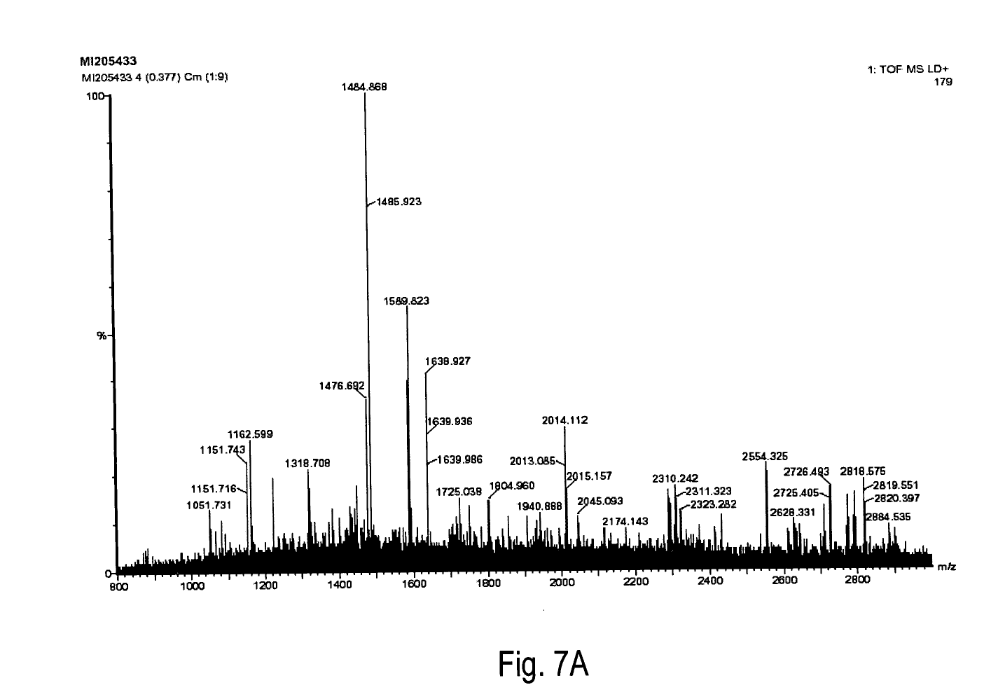

Referring to FIG. 6. 20 protein spots that were equivalent to those positively stained on the 2-DE gel processed with IP using anti-p50 and/or anti-p65 were arbitrarily selected and mapped together using a Typhoon 9200 ImageMaster (Amersham Biosciences) in combination with the ImageMaster 2D Platinum Software, version 5.0 (Amersham Biosciences).

A separate 2-DE gel was run under conditions identical to those used for the silver-stained 2-DE gel of FIG. 6. After SYPRO-Ruby staining, 20 protein spots corresponding to those spots indicated in FIG. 6 were subjected to in-gel trypsin digestion, followed by mass spectrometric analysis using a MALDI-TOF mass spectrometer.

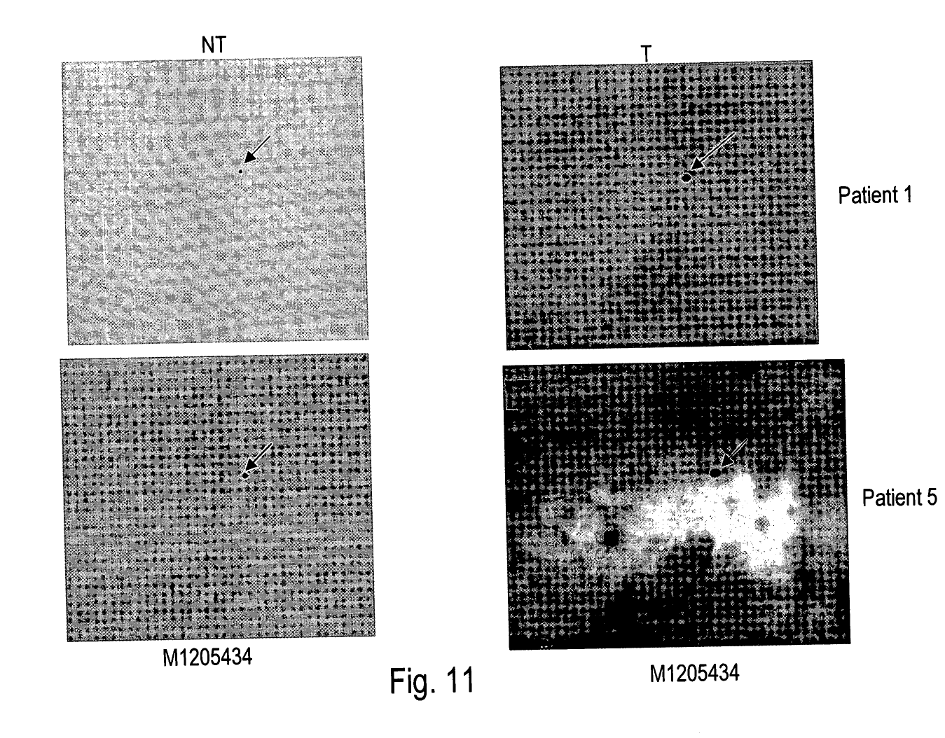

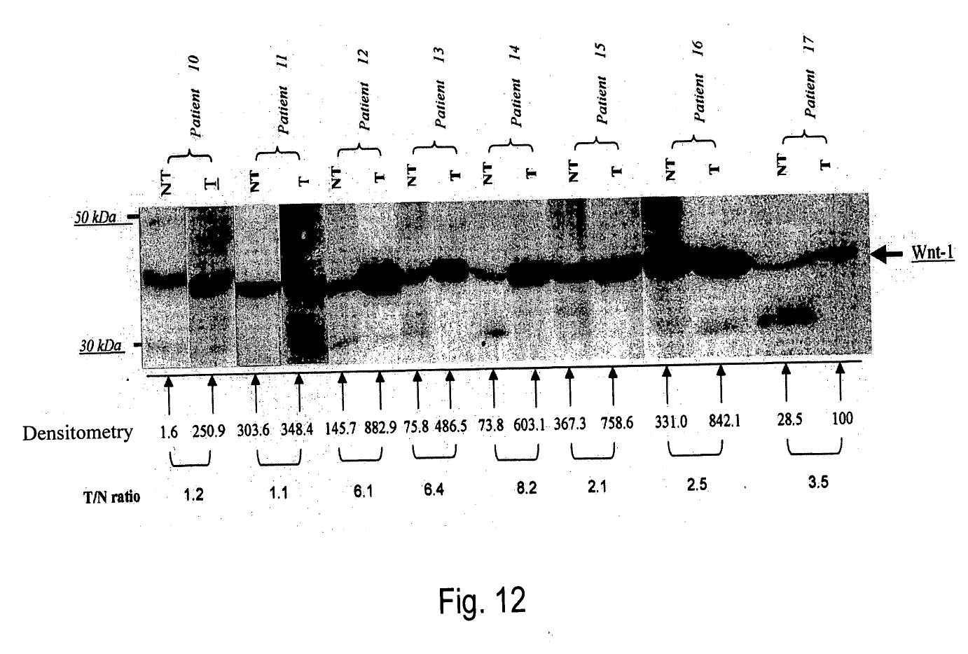

Amongst these analyzed 20 protein spots, a protein spot designated as “M1205434” was suggested to be Wnt-1 protein (NCBI accession no. P04628) based on the database search results by MALDI PMF analysis (see Table 3, infra), experimental results from 2-DE Western blot analysis of the paired-specimens of HCC Patient Nos. 1 and 5 using anti-human Wnt-1 (see FIG. 11), and experimental results from the densitometry analysis on 1-DE Western blot of the specimens from eight additional HCC patients (i.e., Patient Nos. 10-17, see FIG. 12).

Our results revealed constitutive activation of NF-κB in tumor and non-tumor portions of paired liver specimens taken from patients infected with HBV and/or HCV. In addition, to our surprise, it was found that higher levels of NF-κB-associated Wnt-1 protein were detected in tumor portions than in non-tumor portions of paired liver specimens taken from nine patients infected with at least one of HBV and HCV (i.e. HCC patient nos. 1-9 listed in Table 1, infra). In addition, the enhanced expression of Wnt-1 is clinically relevant to the development of HOC in these nine patients infected with at least one of HBV and HCV. The observed enhanced expression of NF-κB-associated Wnt-1 protein was further verified by immunoblot analysis of eight additional paired HCC specimens (i.e., patient nos. 10-17, see FIG. 12). Our studies suggest that enhanced expression of NF-κB-associated Wnt-1 protein may be a common denominator of hepatitis B- and hepatitis C-related hepatocarcinogenesis. Therefore, NF-κB and Wnt-1 protein may be used as potential targets in designing highly effective therapeutic agents for the treatment of HCC and chemoprevention of hepatocarcinogenesis.

Furthermore, intrahepatic spread, early recurrence, portal vein tumor thrombosis, and distant metastases detected within one year after surgery were grouped as poor prognosis. In 42 HCC patients studied so far, high levels of Wnt-1 expression (tumor/nontumor≧2) correlated to poor prognosis in these patients receiving surgical treatment.

Therefore, according to this invention, there is provided a method for determining predisposition to hepatocellular carcinoma in a subject having or suspected to have a hepatic nodule, comprising:
separating a liver specimen taken from the subject into a tumor portion and a non-tumor portion;
detecting the levels of Wnt-1 expression in the tumor portion and the non-tumor portion, respectively;
comparing the detected level of Wnt-1 expression in the tumor portion with that in the non-tumor portion to obtain a value of ratio; and
determining whether or not the subject is predisposed to hepatocellular carcinoma based on the obtained value of ratio, wherein the subject is determined to be predisposed to hepatocellular carcinoma if the obtained value of ratio is greater than 1.

According to this invention, the subject to be examined is a patient who is having liver problem, in particular one infected with at least one of hepatitis B virus and hepatitis C virus.

According to this invention, the hepatic nodule present in the subject may be detected by various physical examinations widely used in clinical practice, e.g., ultrasound, computed tomography (CT), magnetic resonance imaging (MRI), etc. While the hepatic nodule may be clinically detected as such, it may still be necessary to determine whether the hepatic nodule is benign or premalignant or malignant.

According to this invention, the liver specimen is preferably taken from a part of the subject's liver containing a hepatic nodule.

According to this invention, the liver specimen is taken from the subject via a surgical operation selected from segmentectomy and right lobectomy, or via liver biopsy, aspiration or peritoneoscopy. The liver specimen is then separated into a tumor portion and a non-tumor portion based on the gross appearances of liver tissues included in the specimen.

According to the study of this invention, an increase in the detected level of Wnt-1 expression in the tumor portion as compared to that in the non-tumor portion is associated with NF-κB activation.

The levels of Wnt-1 expression may be measured by any means known to those skilled in the art. According to this invention, it is generally preferred to use antibodies, or antibody equivalents, to detect the levels of Wnt-1 expression in liver specimens. However, other methods for detection of Wnt-1 expression can also be used, such as measuring Wnt-1 expression by analysis of mRNA transcripts

Methods for assessing levels of mRNA are well known to those skilled in the art. For example, quantifying mRNA transcript of Wnt-1 gene may be conducted using at least one of the following methodologies: hybridization, Northern blotting, quantitative polymerase chain reaction (PCR) and the like.

Taken as an example, in connection with Northern blotting, a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography. Another common approach for the detection of RNA transcripts is RT-PCR, which involves reverse-transcribing mRNA into cDNA, followed by polymerase chain reaction. Alternatively, mRNA expression can be detected on a DNA array, chip or a microarray. Methods of preparing DNA arrays and their use are well known in the art (see, e.g., U.S. Pat. No. 6,618,679, U.S. Pat. No. 6,379,897, U.S. Pat. No. 6,664,377, U.S. Pat. No. 6,451,536 and U.S. Pat. No. 6,548,257).

In a preferred embodiment of this invention, the levels of Wnt-1 expression are measured by quantifying Wnt-1 protein using at least one of the following methodologies: gel electrophoresis, Western blotting, enzyme immunoassay such as enzyme linked immunosorbent assay (ELISA), radioimmunoassay, immunohistochemistry, proteomics and the like.

In a preferred embodiment of this invention, quantifying Wnt-1 protein is conducted using an antibody-based binding moiety that specifically binds Wnt-1 protein. In a more preferred embodiment of this invention, the antibody-based binding moiety is labeled with a detectable label selected from the group consisting of a radioactive label, a hapten label, a fluorescent label, and an enzymatic label.

The term “antibody-based binding moiety” or “antibody” includes immunoglobulin molecules and immunologically active determinants of immunoglobulin molecules, e.g., molecules that contain an antigen-binding site that specifically binds (immunoreacts with) to Wnt-1 protein. The term “antibody-based binding moiety” is intended to include whole antibodies of any isotype (e.g., IgG, IgA, IgM, IgE, etc.), and includes fragments thereof that are also specifically reactive with Wnt-1 protein.

Antibodies can be fragmented using conventional techniques. Thus, the term “fragment thereof” includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, dabs and single chain antibodies (scFv) containing a VL and VH domain joined by a peptide linker. The scFv's may be covalently or non-covalently linked to form antibodies having two or more binding sites.

The term “antibody-based binding moiety” includes polyclonal, monoclonal, or other purified preparations of antibodies and recombinant antibodies. The term “antibody-based binding moiety” is further intended to include humanized antibodies, bi-specific antibodies, and chimeric molecules having at least one antigen-binding determinant derived from an antibody molecule.

In a preferred embodiment, the antibody-based binding moiety is detectably labeled. As used herein, “Labeled antibody” includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

In the methods of the invention that use antibody-based binding moieties for the detection of Wnt-1, the levels of Wnt-1 protein present in the liver specimens correlate to the intensity of the signal emitted from the detectably labeled antibody.

In one preferred embodiment, the antibody-based binding moiety is detectably labeled by linking the antibody to an enzyme. The enzyme, in turn, when exposed to its substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, β-galactosidase, ribonuclease, urease, catalase, glucose-VI-phosphate dehydrogenase, glucoamylase and acetylcholinesterase. Chemiluminescence is another method that can be used to detect an antibody-based binding moiety.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audoradiography. Isotopes which are particularly useful for the purpose of the present invention are ³H, ³¹P, ³⁵S, ¹⁴C, and ¹²⁵I.

It is also possible to label an antibody with a fluorescent compound. When the fluorescently labeled antibody is exposed to light of the proper wavelength, its presence can be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine. An antibody can also be detectably labeled using fluorescence emitting metals such as ¹⁵²Eu, or others of the lanthanide series. These metals can be attached to the antibody using metal-chelating groups such as diethylenetriaminepentaacetic acid (DTPA) or ethylenediaminetetraacetic acid (EDTA). An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

As mentioned above, the quantity or level of Wnt-1 protein can be detected by immunoassays, such as enzyme-linked immunoabsorbant assay (ELISA), radioimmunoassay (RIA), Immunoradiometric assay (IRMA), Western blotting, or immunohistochemistry. Antibody arrays or protein chips can also be employed (see, e.g., U.S. Pat. Nos. 6,329,209 and 6,365,418; and U.S. patent application Publication Nos. 20030013208A1, 20020155493A1 and 20030017515A1).

Proteomics typically includes the following steps: (1) separation of individual proteins in a sample by 2-D gel electrophoresis (2-D PAGE); (2) identification of the individual proteins recovered from the gel, e.g. by mass spectrometry or N-terminal sequencing; and (3) analysis of the data using bioinformatics. Proteomics methods are valuable supplements to other methods of gene expression profiling, and can be used, alone or in combination with other methods, to detect the products of the biomarker of HCC according to this invention.

Mass spectrometry methods are well known in the art and have been used to quantify and/or identify biomolecules, such as proteins. Therefore, in the method of this invention, Wnt-1 protein may also be detected using mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, EST-MS/MS, etc.). See, for example, U.S. patent application Publication Nos. 20030199001, 20030134304, 20030077616, which are incorporated herein by reference.

According to this invention, there is also provided a method for prognostic evaluation of a subject having or suspected to have hepatocellular carcinoma, comprising:
separating a liver specimen taken from the subject into a tumor portion and a non-tumor portion;
detecting the levels of Wnt-1 expression in the tumor portion and the non-tumor portion, respectively;
comparing the detected level of Wnt-1 expression in the tumor portion with that in the non-tumor portion to obtain a value of ratio; and
evaluating the prognosis of the subject based on the obtained value of ratio, wherein the subject is evaluated to have:
(i) a prognosis of no more than 6 months if the obtained value of ratio is greater than 2;
(ii) a prognosis of 6 to 18 months if the obtained value of ratio is between 1 and 2; or
(iii) a prognosis of at least 18 months if the obtained value of ratio is less than 1.

It is to be understood that the details and particulars concerning the aspect of the method for evaluating the prognosis of a subject having or suspected to have hepatocellular carcinoma will be substantially the same as those of the aspect of the method for determining predisposition to hepatocellular carcinoma in a subject as discussed above, and this means that whenever appropriate, the above statements concerning the liver specimens, the analyses, etc., will apply mutatis mutandis to the aspect of the method for evaluating the prognosis of a subject having or suspected to have hepatocellular carcinoma.

The prognostic method of this invention is useful for determining a proper course of treatment for a patient having or suspected to have HCC. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or to adjust the span of time during which the patient is treated.

This invention also contemplates the manufacture of diagnosis kits for the diagnosis (predisposition) and/or prognosis of HCC in a subject having or suspected to have a hepatic nodule, in which a reagent capable of quantifying the levels of Wnt-1 expression is included.

This invention will be further described by way of the following examples. However, it should be understood that the following examples are solely intended for the purpose of illustration and should not be construed as limiting the invention in practice.

### EXAMPLES

Materials and Methods:

1. Collection of Liver Tissue Samples:

Informed consent was obtained from each of the subjects studied in this invention for the donation of their liver tissues, and the study protocols of this invention conformed to the ethical guidelines of the 1975 Declaration of Helsinki.

Fresh resection specimens of nine HCC patients (patient nos. 1-9) who received surgical treatment of liver tumors in Chi-Mei Medical Center (Tainan, Taiwan) and Chang-Gung Memorial Hospital (Taoyen, Taiwan) were separated into tumor and non-tumor portions immediately after operation. Table 1 summarizes the clinical features of these nine patients. Eight additional paired HCC specimens (patient nos. 10-17), which were acquired from the Tumor and Serum Bank of Chi-Mei Medical Center, were likewise separated into tumor and non-tumor portions and studied for verification. All the HCC specimens were stored at −70° C. prior to experiment.

TABLE 1
The clinical features of nine HCC Patients studied in this invention.

| Patient | Age | | Tumor | | Clinical stage | | | |
|---|---|---|---|---|---|---|---|---|
| No. | (yrs) | Gender | stage | hispathology | (Child-Pugh) | Virus | Background | Operation |
| 1 | 50 | F | I | poor-HCC | A | HCV | CH | Segmentectomy |
| 2 | 60 | M | I | well-HCC | A | HCV | AC | Segmentectomy |
| 3 | 53 | M | I | poor-HCC | A | HCV | AC | Segmentectomy |
| 4 | 62 | F | II | mod-HCC | A | HCV | LC | Segmentectomy |
| 5 | 75 | M | II | well-HCC | A | HBV | CH | Segmentectomy |
| 6 | 49 | M | II | poor-HCC | A | HBV | AC | Segmentectomy |
| 7 | 51 | F | I | well-HCC | A | HBV | LC | Segmentectomy |
| 8 | 75 | M | II | mod-HCC | A | HBV | LC | segmentectomy |
| 9 | 55 | M | II | poor-HCC | B | HBV + HCV | LC | Rt lobectomy |

Note:
M, male;
F, female;
poor-HCC, poorly differentiated hepatocellular carcinoma;
well-HCC, well-differentiated HCC;
mod-HCC, moderately differentiated HCC,
HBV, hepatitis B virus;
HCV, hepatitis C virus;
LC, liver cirrhosis;
AC, active cirrhosis;
CH, chronic hepatitis.

2. Preparation of Protein Samples from Liver Tissues:

Proteins were extracted from the HCC specimens under study. Two groups of experiments were conducted in parallel, one being run individually for each paired-HCC specimens that were previously divided into tumor and non-tumor portions, and the other being carried out on respectively pooled total proteins of the tumor and non-tumor portions of the nine paired-HCC specimens.

The samples were kept on ice at all times during experiment. For each of the individual HCC specimens separated into tumor and non-tumor portions, 0.06-0.08 g of frozen liver tissue was crushed into powder using a chilled stainless-steel mortar and pestle with liquid nitrogen. The resultant tissue powder was mixed with 5 mL of a lysis buffer (7 M urea, 2 M thiourea, 4% CHAPS, 10 mM Tris, and 1 mM PMSF) and the thus-obtained mixture was subjected to homogenization using a Potter-type homogenizer at 4° C. for 1 hr. Unbroken cells and connective tissue were removed from the homogenate by centrifugation at 21,000×g for 3 hrs at 4° C. The supernatant was collected and stored at −70° C. until use.

The protein concentration of the collected supernatant was quantified using a PlusOne™ 2-D Quant Kit (Amersham Biosciences Corp., Piscataway, N.J., USA). A 0.2 g of frozen liver tissue in 5 mL lysis buffer gave a final protein concentration of 5-10 mg/mL. The thus-prepared protein samples were used in the immunoprecipitation and 2-DE electrophoresis experiments.

On the other hand, the protein samples used in the subsequent 1-DE electrophoresis experiment were quantified by Bradford protein assay as previously described.

3. EMSA and Supershift Assay:

Nuclear and cytoplasmic protein extracts were prepared according to standard protocols (S. M. Abmayr and J. L. Workman (1991), Preparation of nuclear and cytoplasmic extracts from mammalian cells. In: Ausubel, F. M., Brent, R., Kingston, R. E., Moore, D. D., Seidman J. G., Smith, J. A., and Struhl, K. eds., Current Protocols in Molecular Biology. New York: John Wiley & Sons; 12.1.1-12.1.9).

Electrophoretic mobility shift assay (EMSA) and supershift assay of NF-κB on the tumor and non-tumor portions of the HCC specimens were performed according to the procedures as described previously (D. I. Tai et al. (2000), Hepatology, 31:656-664; and D. I. Tai et al. (2000), Cancer, 89:2274-2281).

4. Immunoprecipitation of NF-κB-Associated Protein Complexes:

Each of the protein samples (500 μg) prepared from the tumor and non-tumor portions of the nine paired-HCC specimens as described above was in dissolved in 1 mL of a rehydration buffer (7 M urea, 2 M Thiourea, 4% CHAPS, 0.5% IPG buffer and a few drops of Bromophenol blue), followed by mixing with 10 μL of either anti-NF-κB p50 antibody (anti-p50) or anti-NF-κB p65 antibody (anti-p65)(Biogenesis, Poole, UK) for 1.5 hrs at 4° C. The resultant immunocomplexes, i.e., the NF-κB-associated protein complexes, were collected by Protein A Sepharose™ CL-4B beads (Amersham Biosciences) according to the manufacturer's instructions.

5. Two-Dimensional Electrophoresis (2-DE):

The immunocomplexes collected from the tumor portions of the specimens of the nine HCC patients listed in Table 1, supra, were pooled and then subjected to a two-dimensional electrophoresis (2-DE) analysis using a 12.5% polyacrylamide gel under denatured conditions according to standard protocols (P. H. O'Farrell (1975), J. Bio. Chem., 250:4007-4021) and the detailed procedures as described previously (C. L. Lee et al. (2003), Proteomics, 3:2472-2486).

In the experiments of this invention, in addition to the NF-κB-associated protein complexes collected from immunoprecipitation, total proteins from the tumor and non-tumor portions of the specimens of the 9 HCC patients listed in Table 1, supra, were examined in parallel.

6. Silver Staining:

A modified silver staining method, which is compatible with mass spectrometric analysis to provide protein spot visualization, was used in the study protocols of this invention (C. L. Lee et al., (2003), Proteomics, 3:2472-2486). Briefly, the polyacrylamide gels after 2-DE were fixed in 50% methanol/10% acetic acid in water for 30 minutes, followed by incubation in 5% methanol for 15 minutes. Thereafter, the 2-DE gels were washed three times with Milli-O water for five minutes each and then sensitized with freshly prepared 0.02% sodium thiosulphate for exactly two minutes, followed by washing three times with Milli-Q water for 30 seconds each. Thereafter, the 2-DE gels were treated with 0.2% silver nitrate for 25 minutes and rinsed three times with Milli-Q water for one minute each. The 2-DE gels were then immersed in a developer solution comprising 3% sodium carbonate, 0.018% formaldehyde and 0.02% sodium thiosulphate. The desired intensity of staining was achieved after immersing the 2-DE gels in the developer solution for three to four minutes. The development was stopped by the addition of 1.4% sodium EDTA for 10 minutes, and the 2-DE gels were then rinsed twice with Milli-Q water for two minutes each.

7. SYPRO-Ruby Staining:

2-DE electrophoresis was run on a separate 12.5% polyacrylamide gel under conditions identical to those described above. Thereafter, the gel was fixed in 10% methanol/7% acetic acid in water for 30 minutes, followed by washing three times each with water for five minutes. To obtain the maximum sensitivity, the gel was incubated with a SYPRO-Ruby solution (Molecular Probes, Eugene, Oreg., USA) according to the manufacturer's instructions for at least three hrs. To reduce background fluorescence and to increase sensitivity, the stained gel was washed with 10% methanol/7% acetic acid in water for 30 minutes.

8. Image Recording and Analysis:

After silver staining, the 2-DE gels were rinsed twice with water for five minutes each, before being scanned on a Typhoon 9200 ImageMaster (Amersham Biosciences). The image analysis and 2-D gel proteome database management were done using the ImageMaster 2D Platinum Software, version 5.0 (Amersham Biosciences). The theoretical Mr and pI values of the 2-DE markers were used to calibrate the Mr and pI values of the protein spots on the 2-DE gels. Intensity levels were normalized between gels as a proportion of total protein intensity detected for the entire gel and the protein quantity of each spot calculated by integrating density over the spot area (C. L. Lee et al. (2003), Proteomics, 3: 2472-2486; M. J. Hubbard and N. J. McHugh (2000), Electrophoresis, 21: 3785-3796).

9. Mass Spectrometric Analysis:

The SYPRO-Ruby-stained 2-DE gel was subjected to in-gel trypsin digestion. Thereafter, Tryptic peptides were obtained from selected protein spots on the stained 2-DE gel and then subjected to MALDI peptide mass fingerprinting (PMF) using a matrix-assisted laser desorption/ionization quadrupole-time of flight (MALDI-Q-TOF) mass spectrometer (M@LDI™; Micromass, Manchester, UK) operated in reflectron positive ion mode as described previously (C. L. Lee et al. (2003), Proteomics, 3:2472-2486; J. Kim et al. (2002), Electrophoresis, 23:4142-4156). Briefly, samples were spotted onto a 96-well format MALDI target plate using a saturated matrix solution of α-cyano-4-hydroxycinnamic acid (CHCA) in 60% ACN/1% TFA. The instrument was externally calibrated with standard peptide mixtures and further adjusted with the lock mass feature using adenocorticotropic hormone (ACTH) as the near-point calibrant. Mass spectra were acquired for the mass range of 900-3000 Da and automatically processed by the ProteinLynx™ software for PMF searches against the SWISS-PROT database employing the MASCOT program (A. I. Nesvizhskii and R. Aebersold (2004), Drug Disc. Today, 9:173-181). The search parameters allowed for one missed cleavage, oxidation of methionine, N-terminal acetylation, and carboxyamido-methylation of cysteine. Positive identification of proteins required at least five matching peptide masses with 50 ppm or better mass accuracy.

10. One-DE and 2-DE Western Blot Analysis of Wnt-1 Protein:

Analytical 1-DE and 2-DE gels were electrotransferred onto PVDF membranes (Hybond P, Amersham Biosciences) for Western blot analysis of Wnt-1 protein according to the standard procedures, in which the used primary antibody was biotin-conjugated rabbit anti-human Wnt-1 antibody (ZYMED Lab. Inc., CA), and the used secondary antibody was HRP-linked mouse anti-rabbit IgG (Amersham Pharmacia Biotech, N.J., USA). Thereafter, the PVDF membranes were treated with an enhanced chemiluminescence detection system (ECLplus, Amersham Biosciences), followed by exposure to autoradiography films for 3-15 minutes. The expression level of Wnt-1 protein was semi-quantitatively estimated on the films using the ImageMaster TotalLab, Version 2.01 (Amersham Pharmacia Biotech, NJ, USA).

Results:

1. Constitutive Activation of NF-κB in HBV- and HCV-Related HCC Tumor and Non-Tumor Tissues:

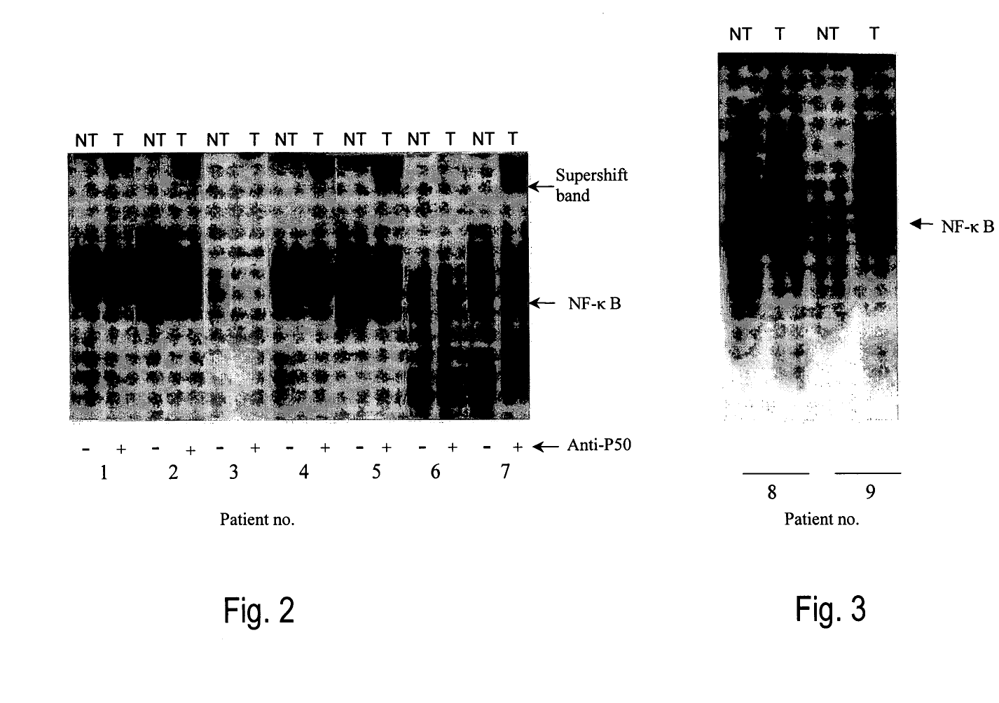

Activation of NF-κB in tumor and non-tumor portions of HBV- and HCV-related HCC liver specimens was analyzed by EMSA, alone or in combination with the supershift assay. Specifically, nuclear protein samples from the tumor and non-tumor portions of the specimens of HCC patient nos. 1-9 listed in Table 1 were subjected to EMSA, alone or in combination supershift assay using anti-p50 as a probe for NF-κB. FIG. 2 shows the EMSA and supershift assay results of nuclear protein samples from the tumor and non-tumor portions of the specimens of HCC patient nos. 1-7 as probed with anti-p50, whereas FIG. 3 shows the EMSA results of nuclear protein samples from the tumor and non-tumor portions of the specimens of HCC patient nos. 8 and 9. Another EMSA experiment was conducted using one paired-HCC tumor and non-tumor tissues, one normal liver control from liver biopsy during cholecystectomy of gallbladder stones, and tumor tissues from five of the eight additional specimens of HCC patient nos. 10-17, for further verification of the study of this invention (data not shown).

The specificity of the shifted band was ascertained by competition studies with a mutant probe and a 50-fold excess amount of a wild-type cold probe used in the EMSA experiment conducted according to the procedures described in D. I. Tai et al. (2000), Hepatology, 31:656-664. Supershift studies showed that the activated NF-κB bands in HBV- and HCV-infected livers undergo a supershift with anti-p50 (see FIG. 2). Supershift experiment with anti-p65 revealed similar results (data not shown). The shift band of both HBV- and HCV-infected liver was almost totally abolished by the excess wild-type cold probe (data not shown) but was not changed when competed with the mutant probe (data not shown).

It has been reported in literature that numerous factors or proteins are associated with NF-κB activation (W. C. Sha (1998), J. Exp. Med., 187:143-146; Q. Li and I. M. Verma (2002), Nature Rev. Immunol., 2:725-734; D. Hanahan and R. A. Weinberg (2000), Cell, 100:57-70; M. Karin et al. (2002), Nature Rev. Cancer, 2:301-310; E. Pikarsky et al. (2004), Nature, 431: 461-466; A. Lin and M. Karin (2003), Semin. Cancer Biol., 13: 107-114). Thus, NF-κB activation in tumor portions is not necessarily more prominent than that in non-tumor portions of the same patient. The EMSA and supershift assay results shown in FIGS. 2 and 3 reveal that constitutive activation of NF-κB is present in the tumor and non-tumor portions of the paired specimens of the HBV- and/or HCV-infected patient nos. 1-9 listed in Table 1.

2. Two-DE of NF-κB-Associated Protein Complexes with or without Immunoprecipitation (IP):

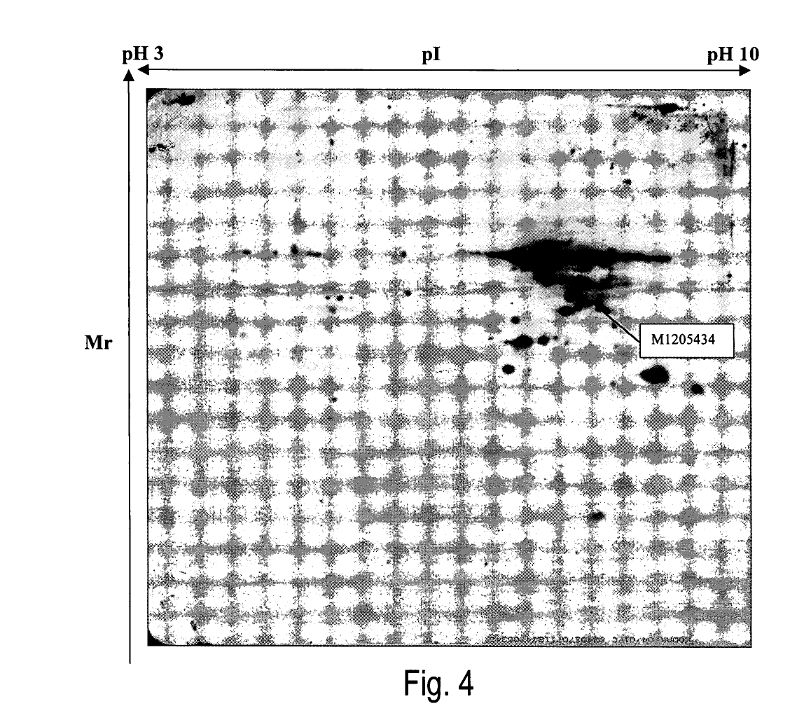

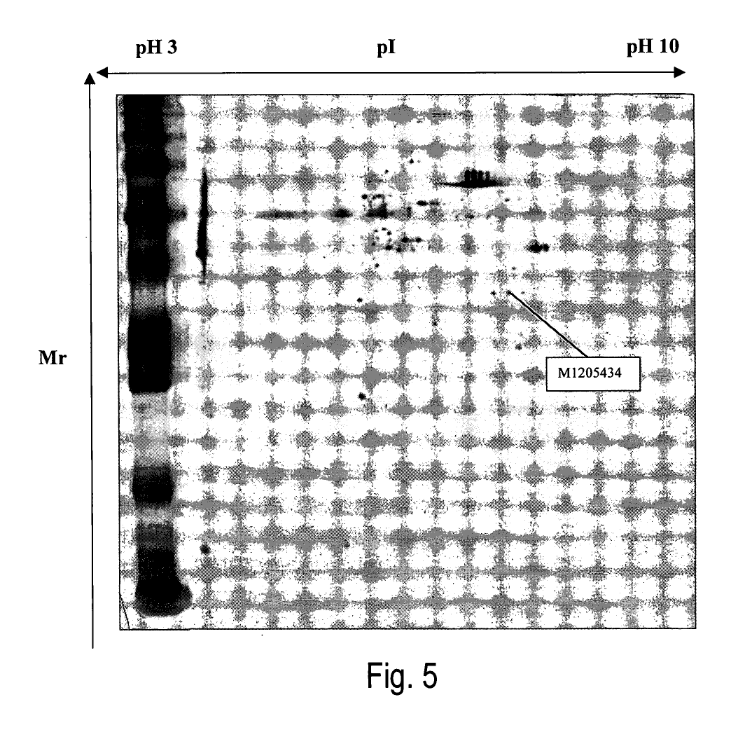

Proteins extracted from the tumor portions of the specimens of the nine HCC patients listed in Table 1 were respectively subjected to immunoprecipitation (IP) using either anti-p50 or anti-p65, and the resultant NF-κB-associated protein complexes in the protein samples of the 9 HCC patients were collected and pooled together to run 2-DE analysis, followed by silver staining. FIG. 4 shows the proteome profile of NF-κB-associated protein complexes probed by anti-p50, whereas FIG. 5 shows the proteome profile of NF-κB-associated protein complexes probed by anti-p65.

Another experiment was parallelly conducted by directly subjecting pooled total proteins from the tumor portions of the specimens of the nine HCC patients listed in Table 1 to 2-DE analysis without IP, followed by silver staining. The results are shown in FIG. 6.

Referring to FIG. 6, 20 spots that were equivalent to those positively silver-stained on the 2-DE gels processed with IP using anti-p50 and/or anti-p65 were arbitrarily selected and mapped together. These 20 spots were selected for analysis in the subsequent experiments.

3. Mass Spectrometric Analysis of Protein Spots:

A separate 2-DE gel was run under conditions identical to those used for the silver-stained 2-DE gel. After SYPRO-Ruby staining, 20 protein spots corresponding to those spots indicated in FIG. 6 were subjected to in-gel trypsin digestion, followed by mass spectrometric analysis using a MALDI-TOF mass spectrometer.

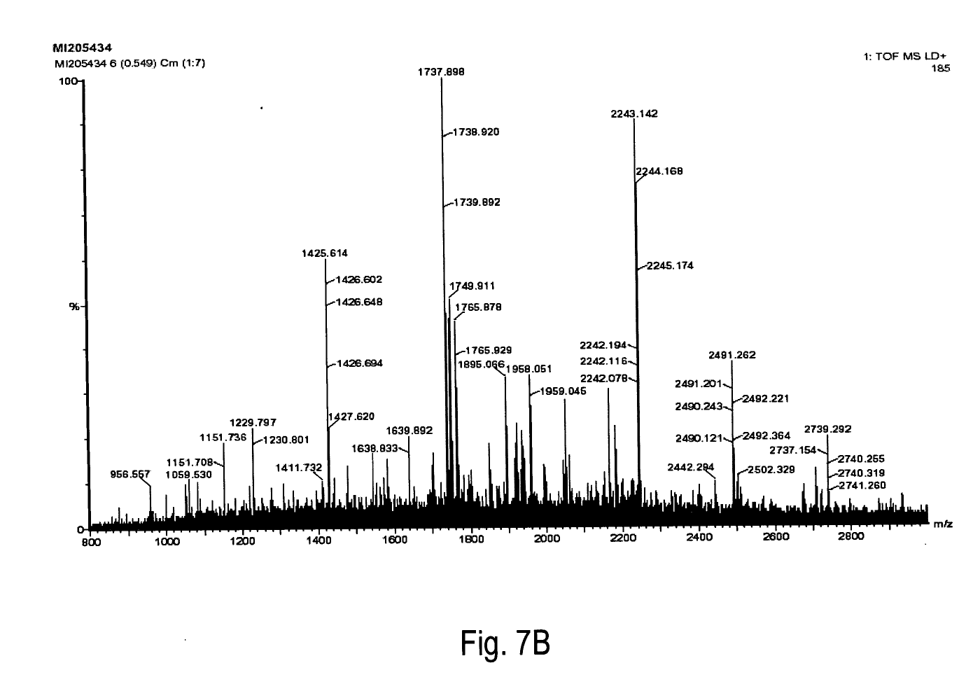

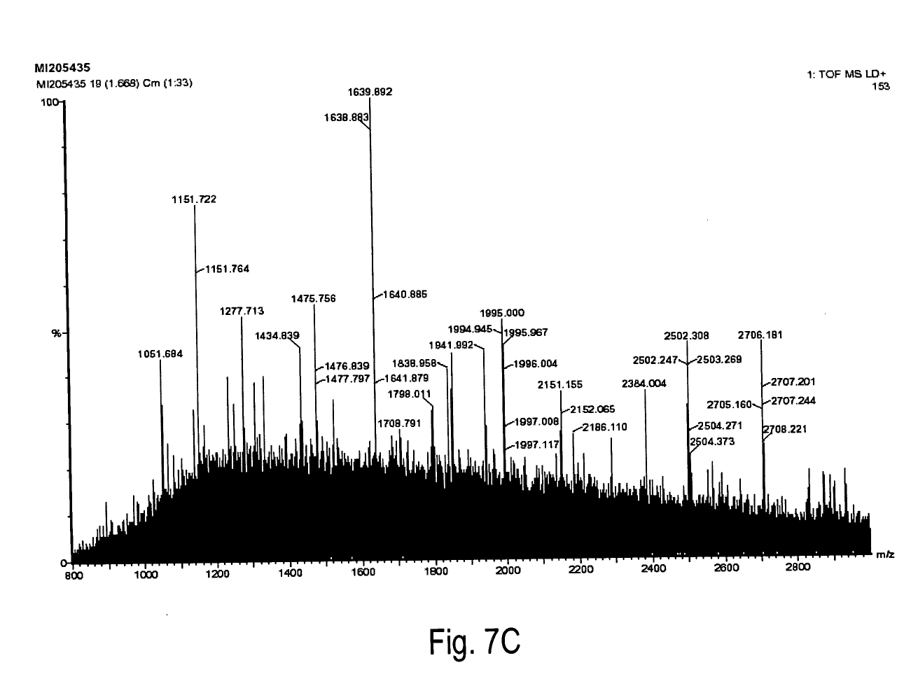

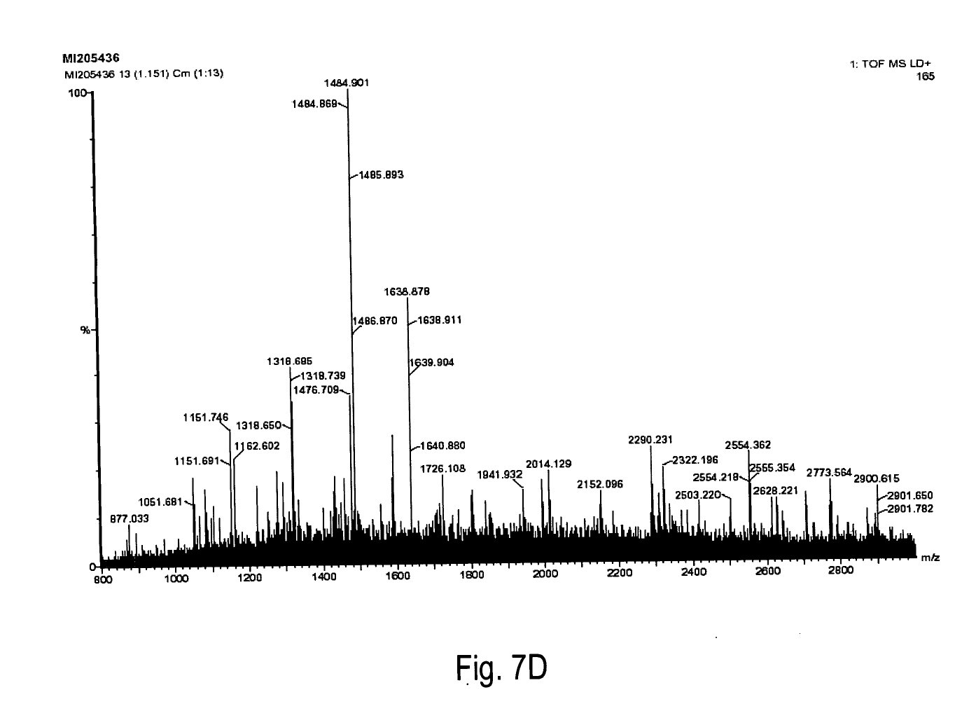

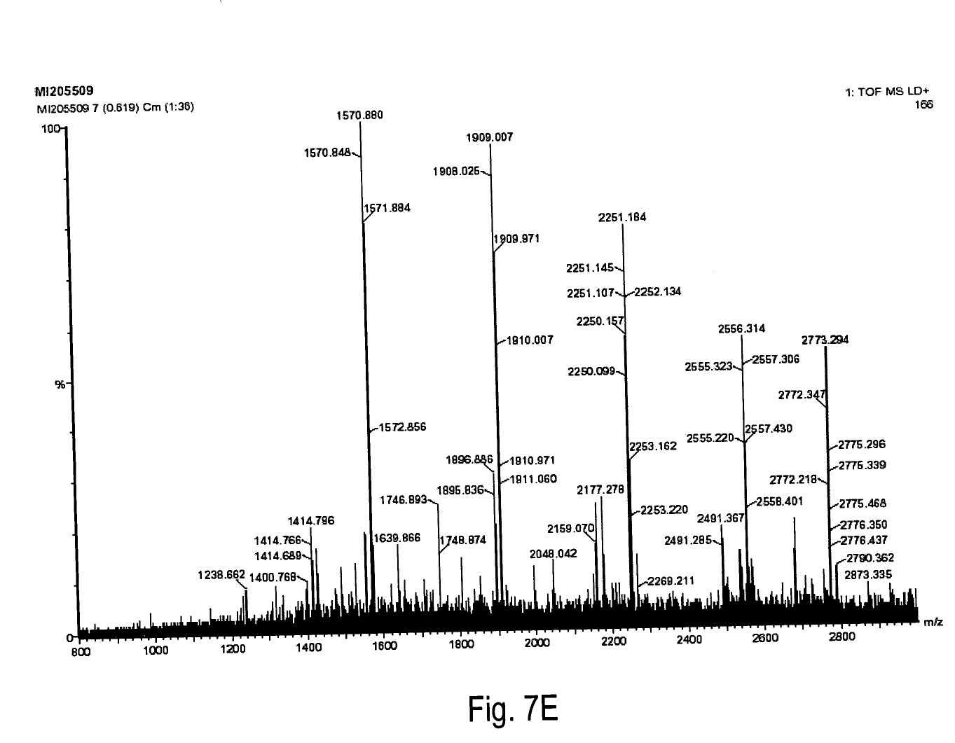

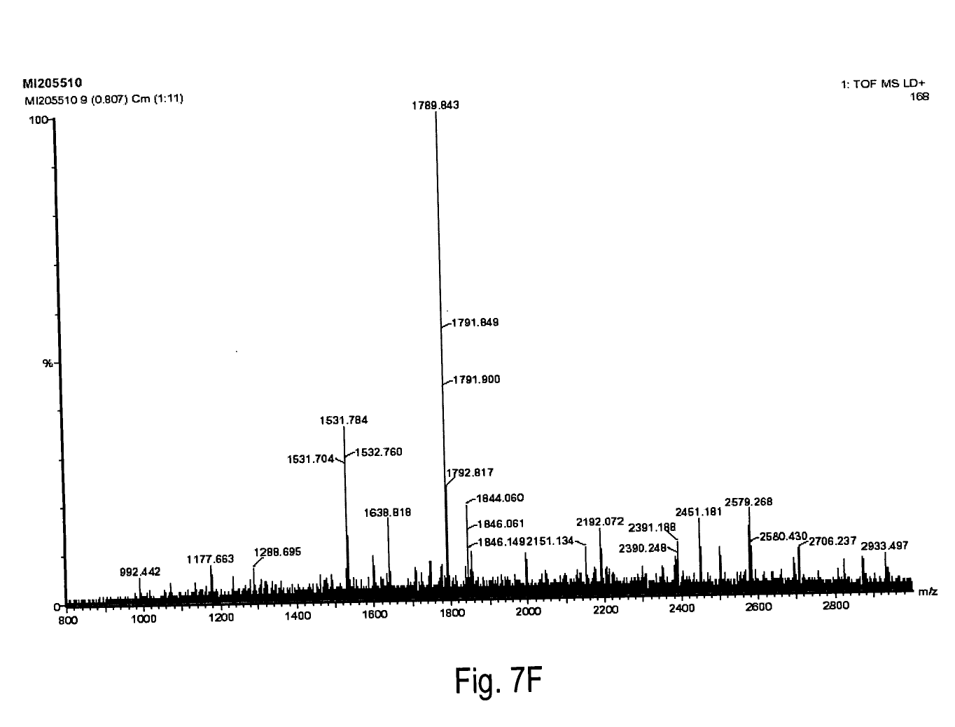

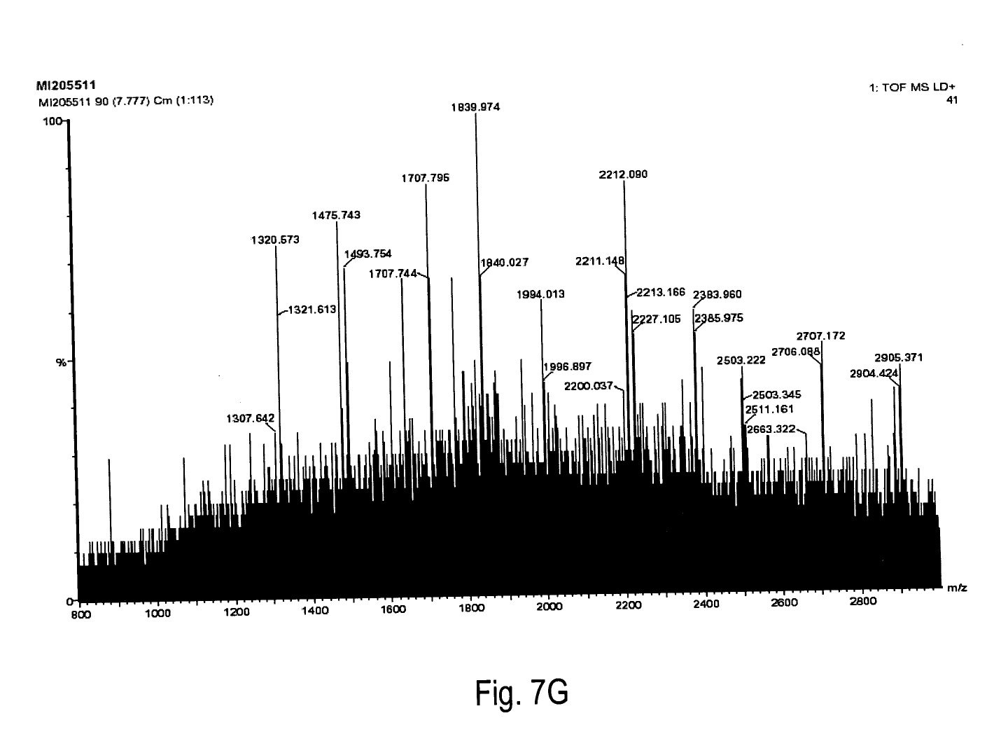

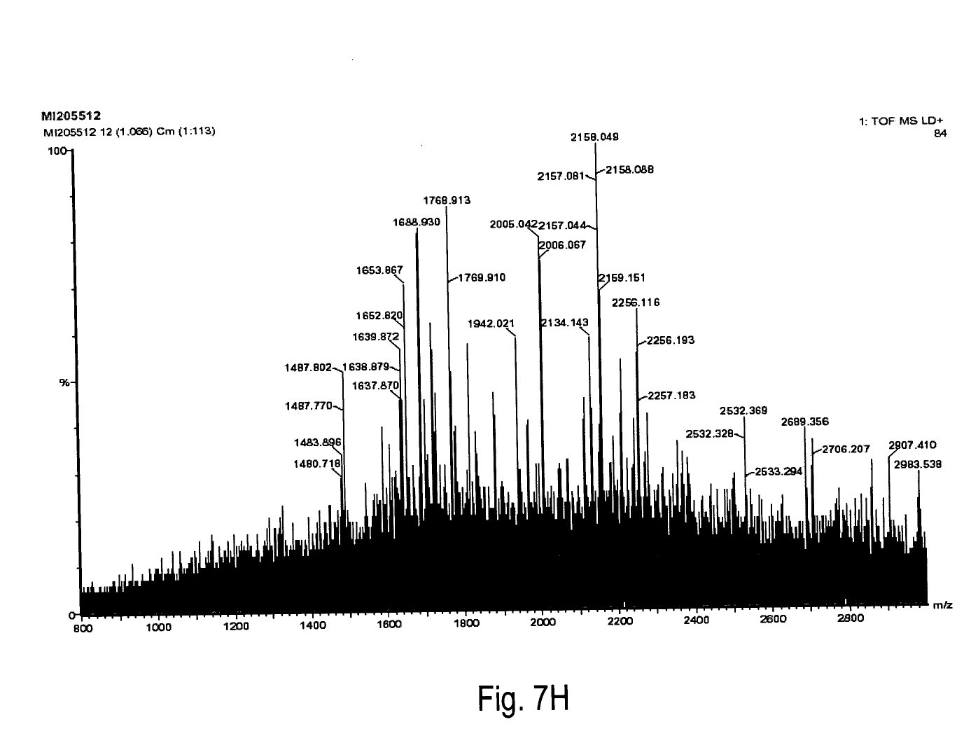

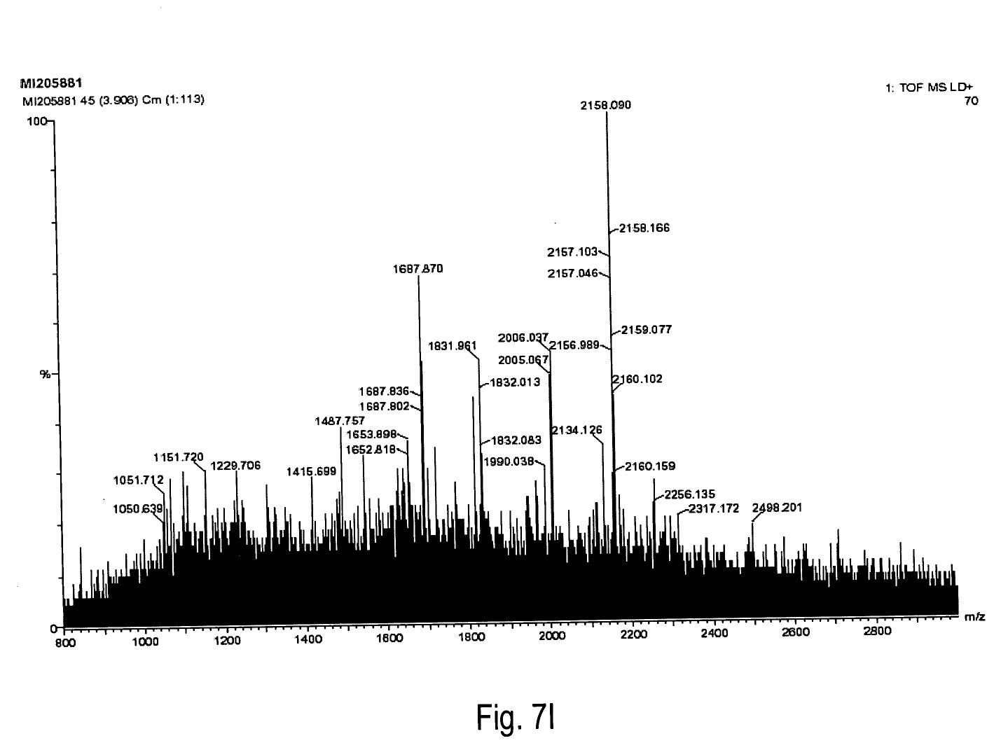

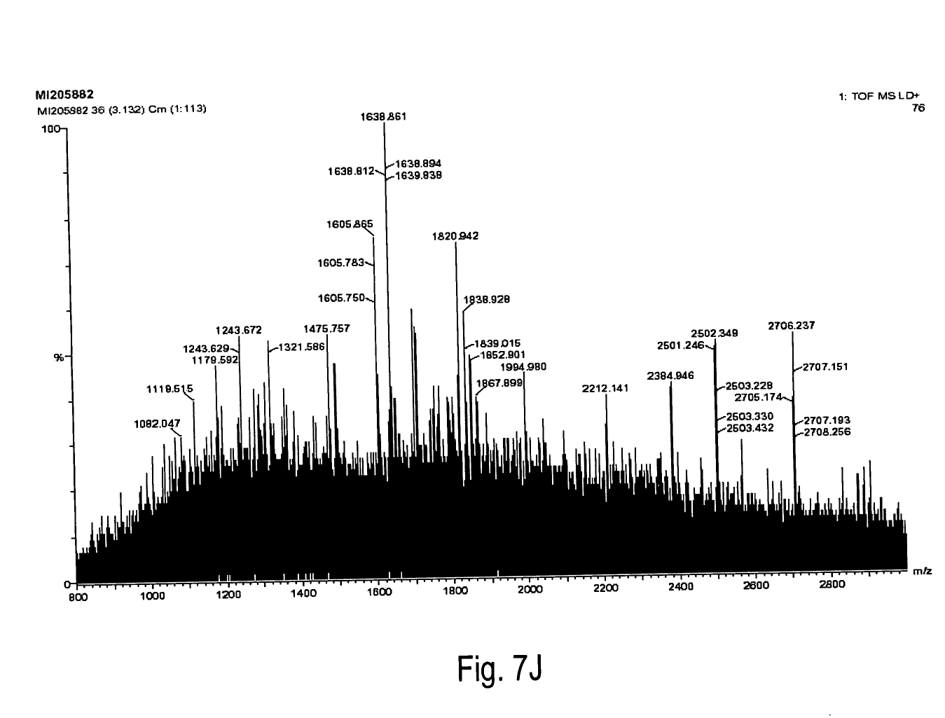

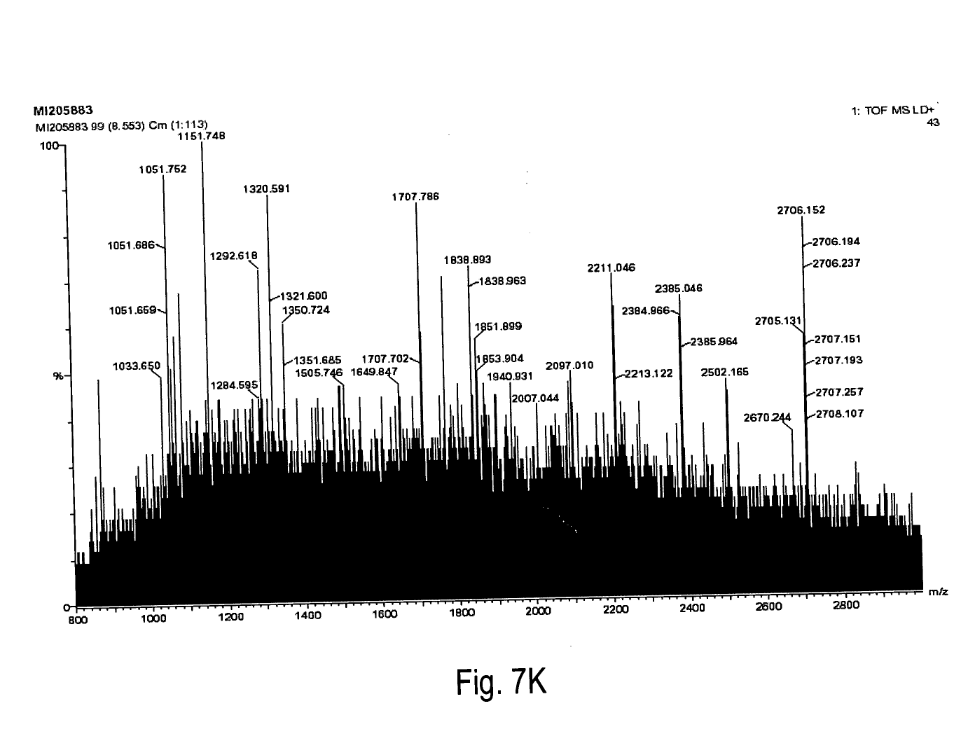

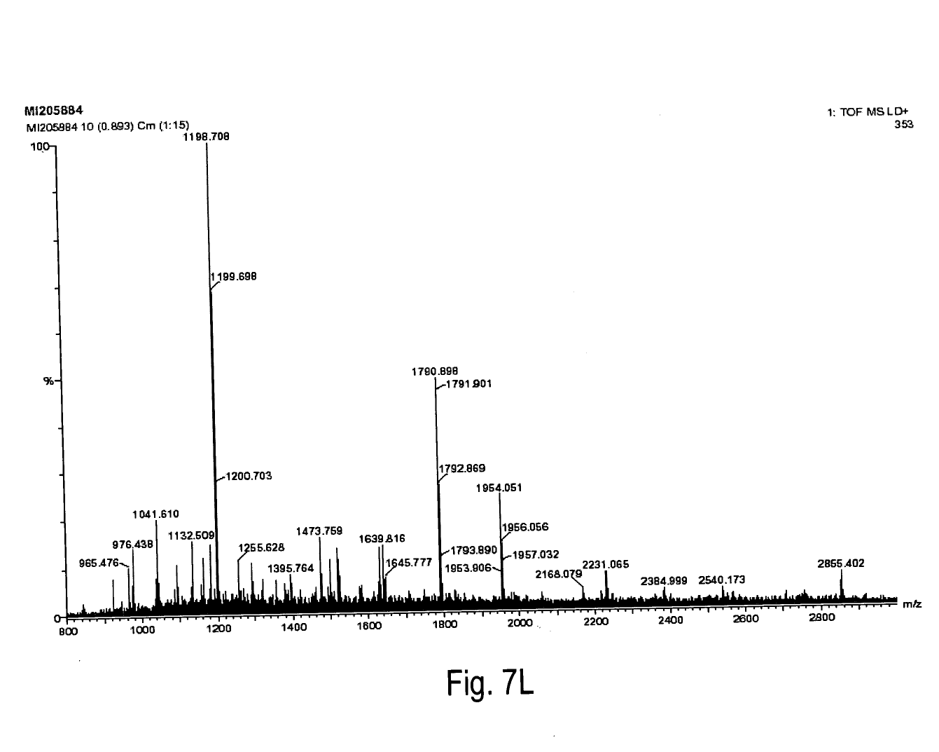

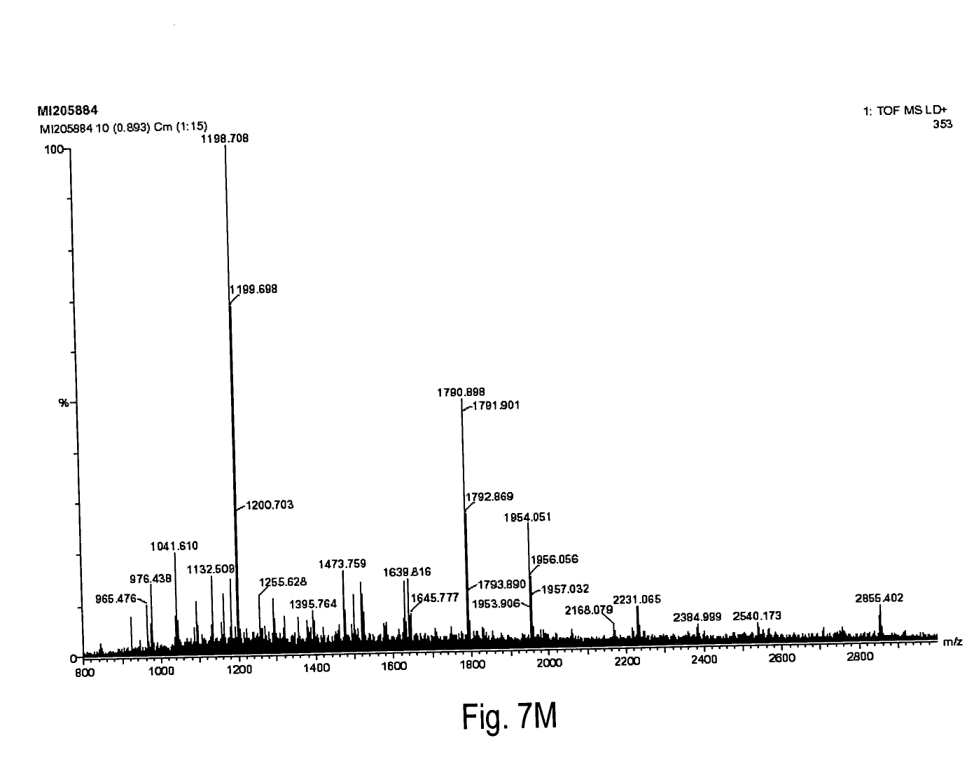

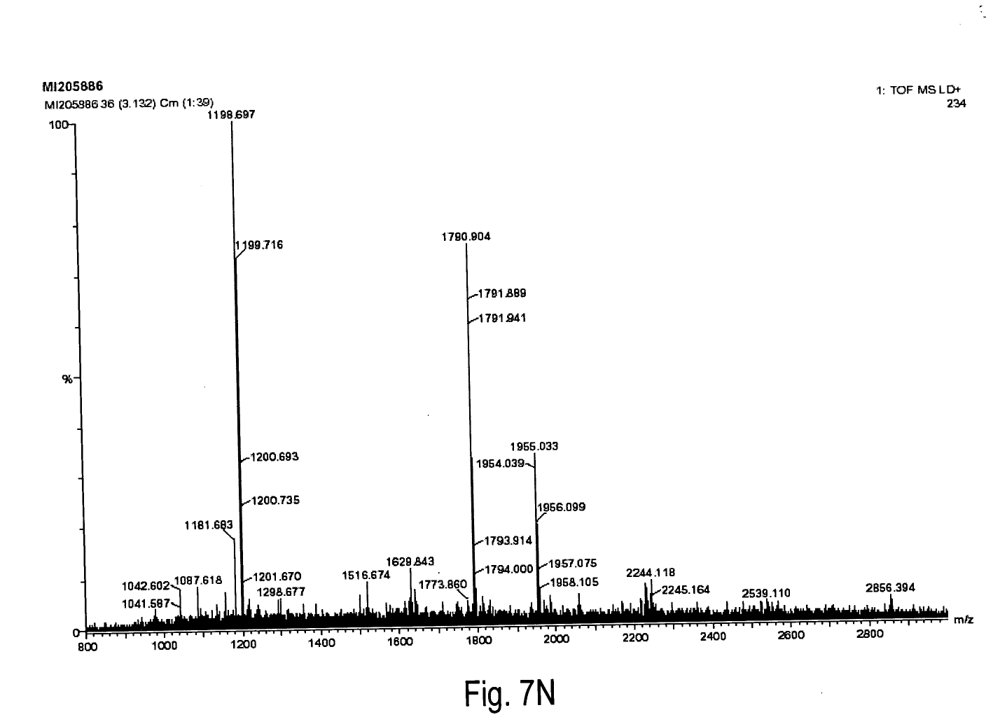

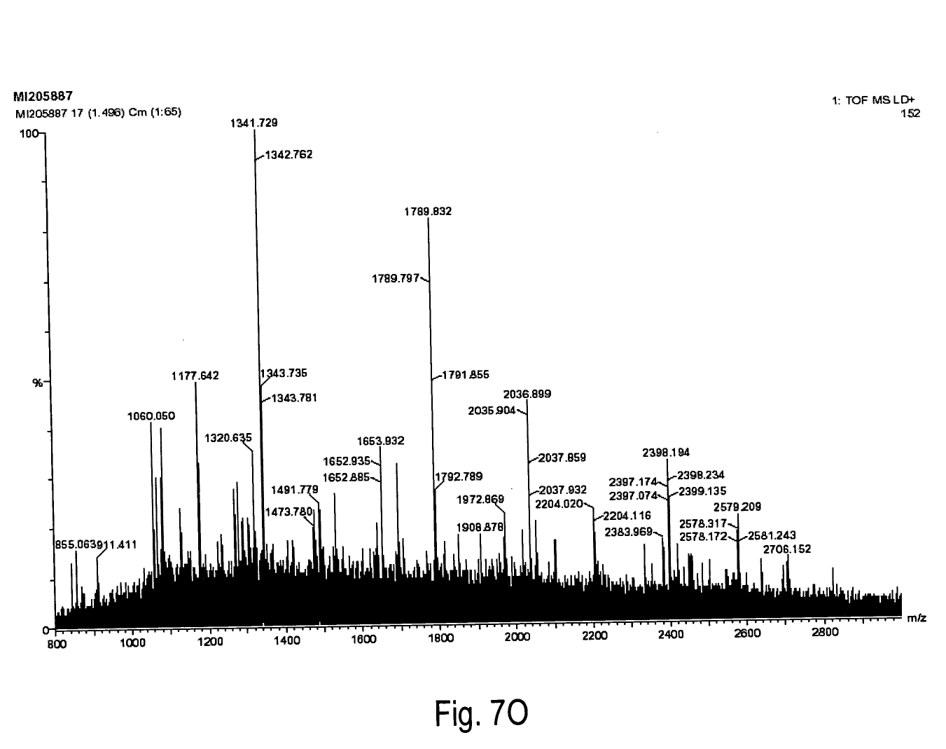

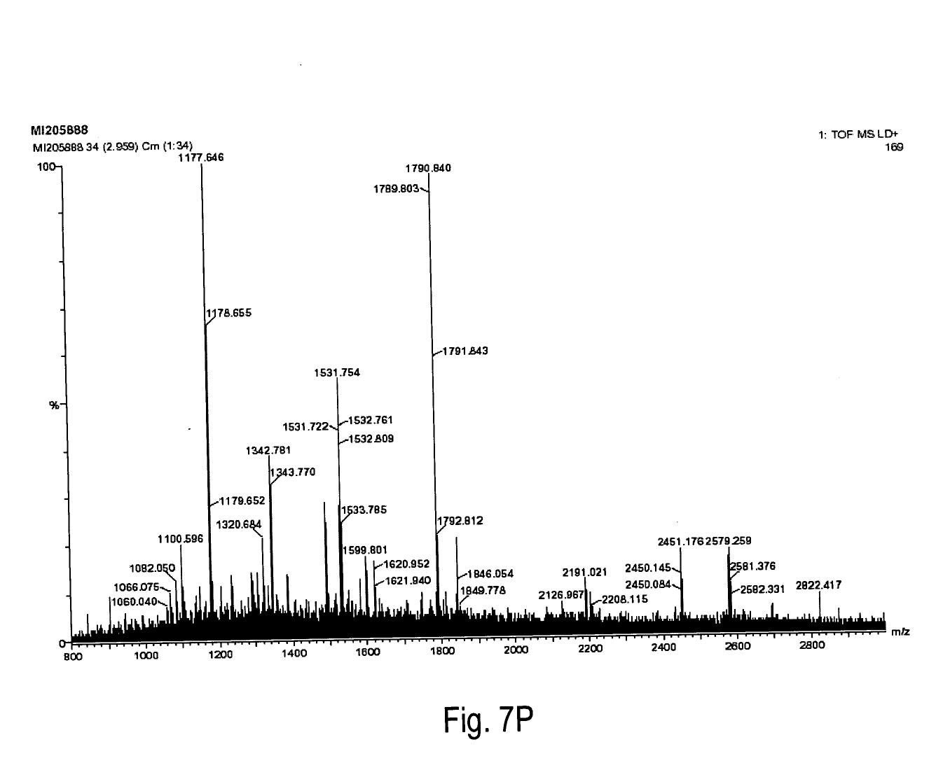

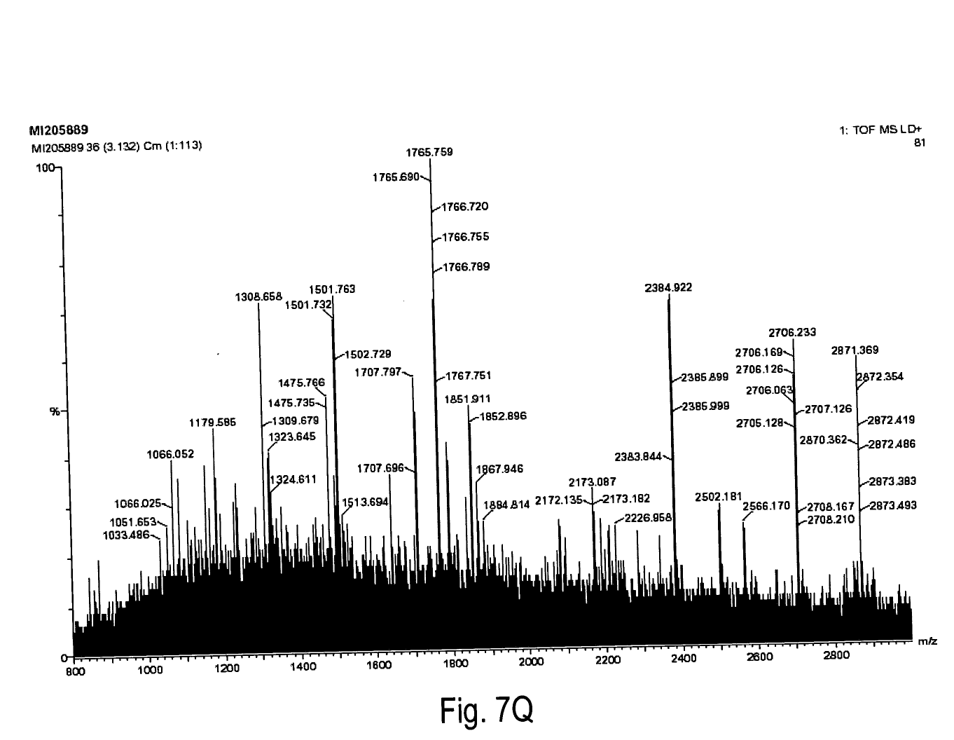

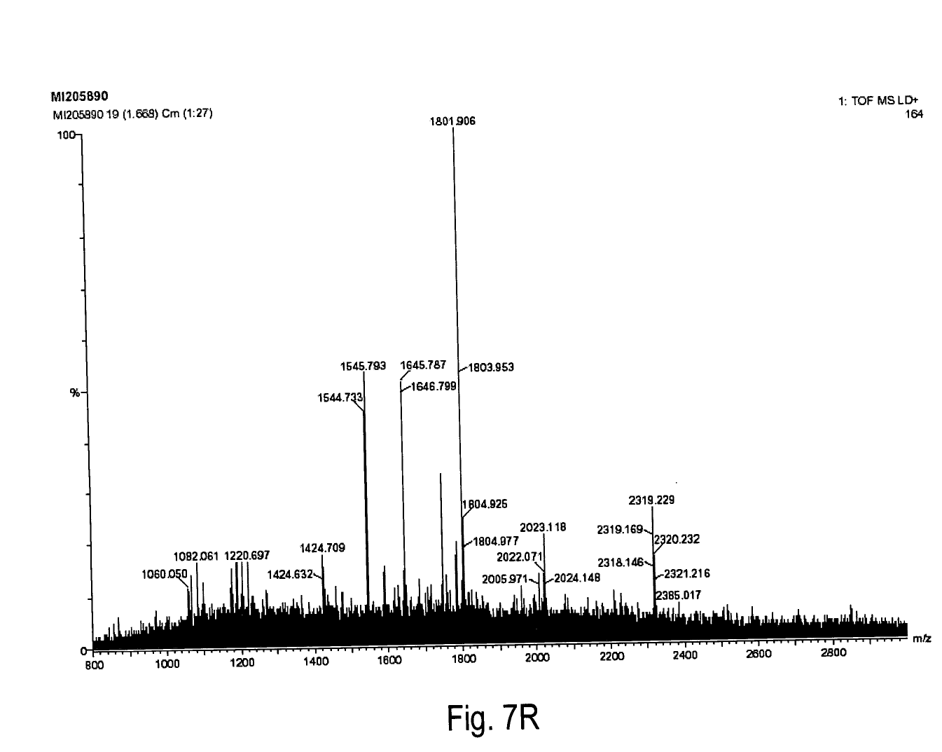

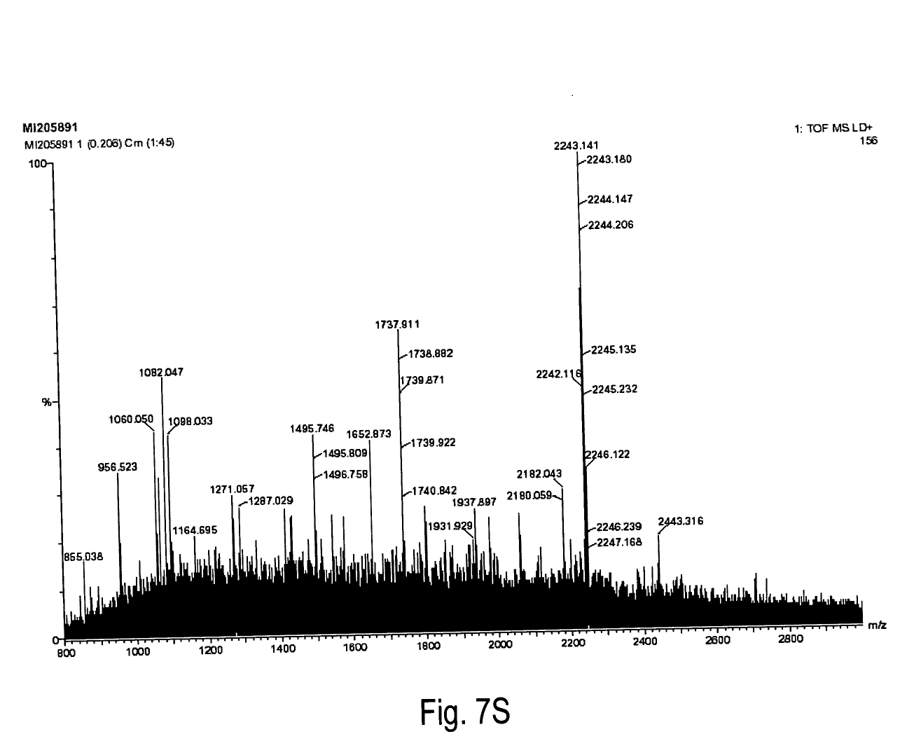

FIG. 7 shows the mass spectrometric analysis results of these 20 selected spots. In addition, database search results obtained after mass spectrometric analysis of these 20 protein spots ruled out the possibility that some spots on the 2-DE profiles of IP proteins might be the derivatives of heavy chain or light chain of anti-NF-κB antibodies (data not shown).

4. Volume Comparison and Immunoblot Analysis of Spot MI205434 in Tumor And Non-Tumor Portions of the Liver Specimens from HBV- and/or HCV-Related HCC Patients:

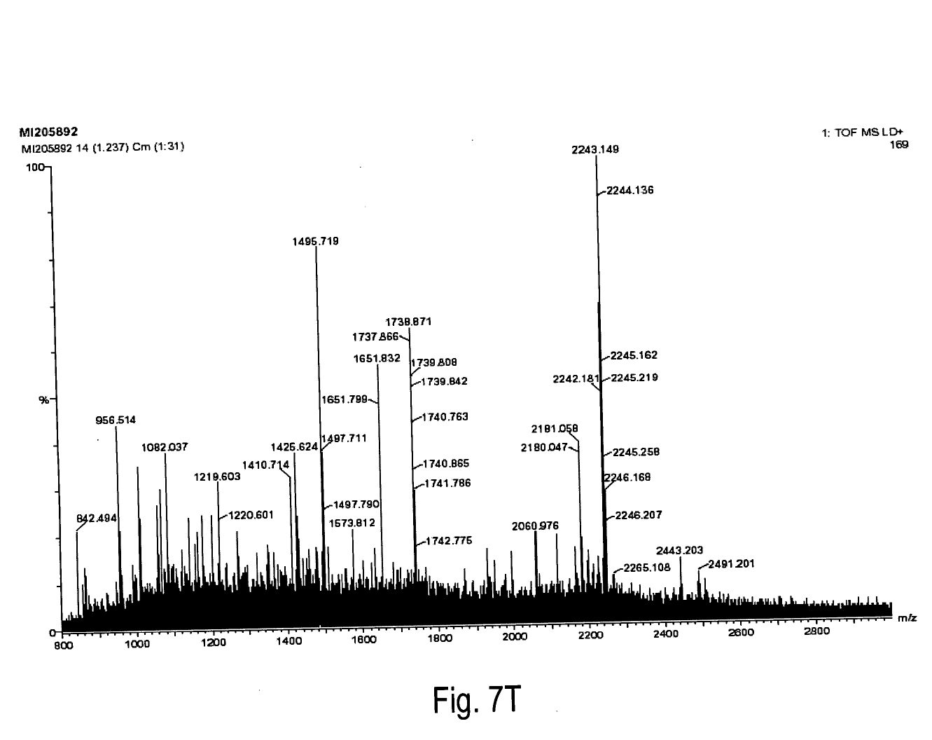

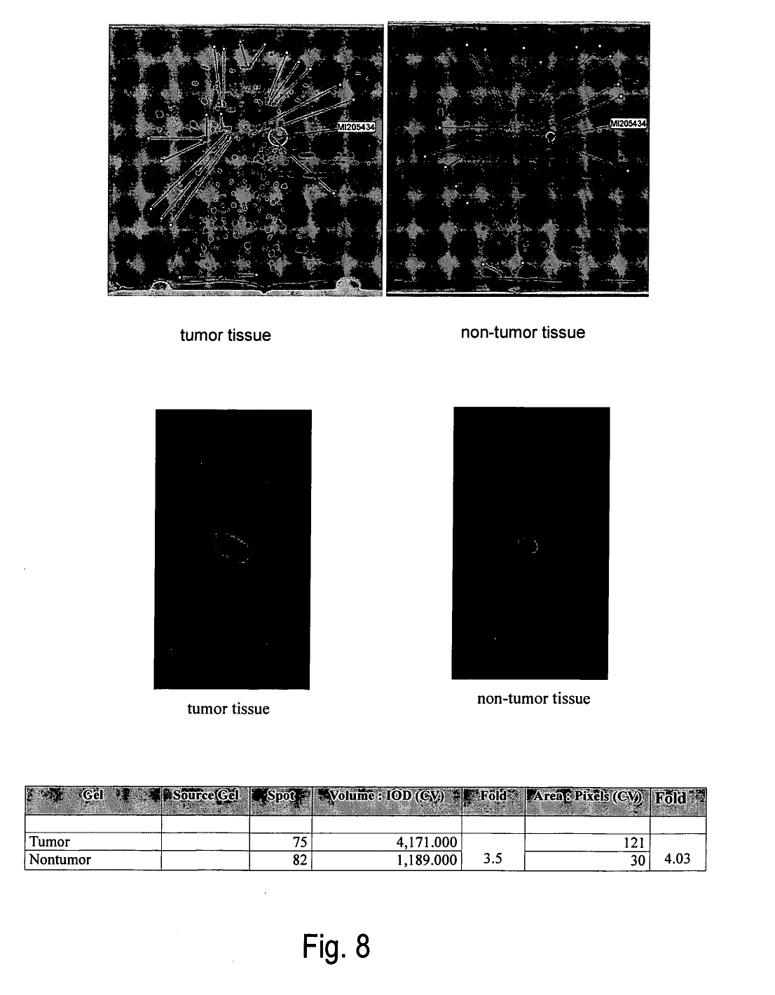

The 20 protein spots selected from the silver-stained 2-DE gel of FIG. 6 were measured semi-quantitatively. It was surprising to find that there were differences, in terms of the expression levels of said protein spots, between the tumor and non-tumor portions of the nine paired-HCC specimens. In particular, for spot MI205434, the measured volume thereof was significantly higher in the tumor portion than in the non-tumor portion by at least two-fold amongst 7 of the nine paired-HCC specimens. FIGS. 8-10 respectively show the volume comparison results of spot MI205434 between the tumor and non-tumor portions of the specimens of HCC patient nos. 1, 5 and 9 listed in Table 1, supra.

Table 2 shows the ratio of the measured volume of spot MI205434 in the tumor portion to that in the non-tumor portion of each of the nine paired-HCC specimens. A highest increase (11.4 times) of the expression of spot MI205434 (i.e., Wnt-1 protein) in the tumor portion versus that in the non-tumor portion was observed in HCC Patient No. 9, who was afflicted with hepatitis B and hepatitis C.

TABLE 2
The ratio of the measured volume of spot MI205434 in the
tumor portion to that in the non-tumor portion of each of the
nine paired-HCC specimens.

| | HCC Patient No. | | | | | | | | |
|---|---|---|---|---|---|---|---|---|---|
| | 1 | 2 | 3 | 4 | 5 | 6 | 7 | 8 | 9 |
| Tumor:non-tumor | 3.5* | 0.9 | 3.0 | 2.6 | 1.4* | 2.7 | 2.1 | 2.2 | 11.4** |

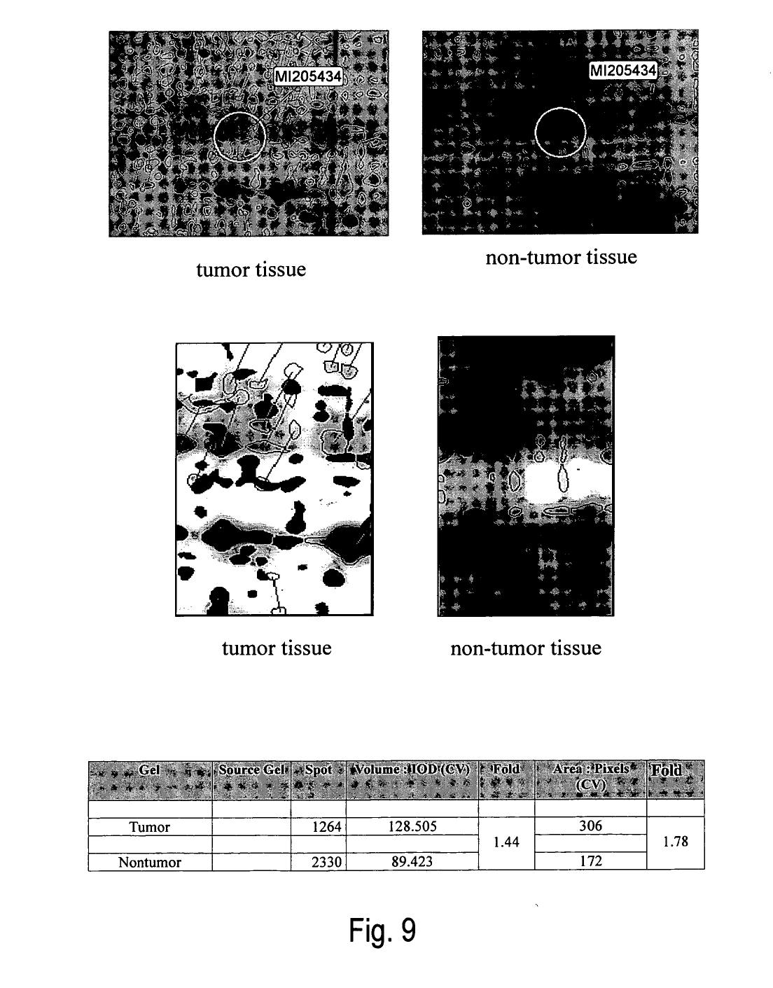

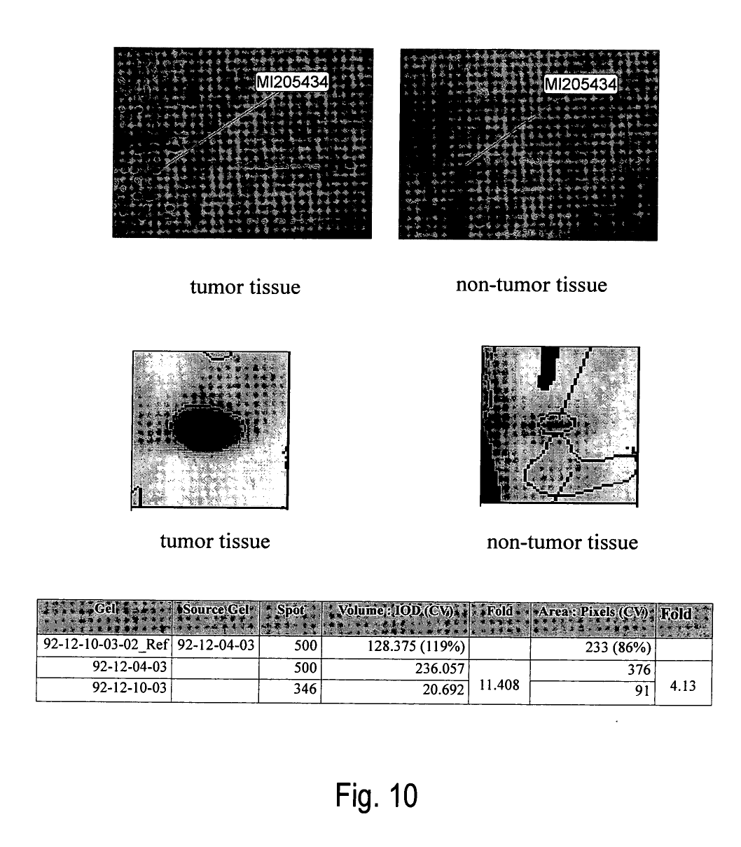

*The ratios were calculated based on the measured volumes of spot MI205434 in the tumor portions to those in the non-tumor portions of the paired-specimens of HCC Patient Nos. 1 and 5 as shown in FIGS. 8 and 9, respectively.
**The ratio was calculated based on the measured volume of spot MI205434 in the tumor portion to that in the non-tumor portion of the paired-specimen of HCC Patient No. 9 as shown in FIG. 10.

The database search results of spot MI205434 by MALDI peptide mass fingerprint (PMF) analysis showed that there were several protein candidates for spot MI205434 (data not shown). However, integration of the database search results by MALDI PMF analysis (see Table 3), experimental results from 2-DE Western blot analysis of the paired-specimens of HCC Patient Nos. 1 and 5 using anti-human Wnt-1 (see FIG. 11), and experimental results from the densitometry analysis on 1-DE Western blot of the specimens from eight additional HCC patients (Patient Nos. 10-17)(see FIG. 12) suggested that the most likely candidate protein for spot MI205434 was Wnt-1 protein.

TABLE 3

Database search results of peptide mass fingerprint
for spot MI205434.*

| | |
|---|---|
| 1 | MGLWALLPGW VSATLLLALA ALPAALAANS SGRWWGIVNV ASSTNLLTDS |
| 51 | KSLQLVLEPS LQLLSRKQRR LIRQNPGILH SVSGGLQSAV RECKWQFRNR |
| 101 | RWNCPTAPGP HLFGKIVNRG CRETAFIFAI TSAGVTHSVA RSCSEGSIES |
| 151 | CTCDYRRRGP GGPDWHWGGC SDNIDFGRLF GREFVDSGEK GRDLRFLMNL |
| 201 | HNNEAGRTTV FSEMRQECKC HGMSGSCTVR TCWMRLPTLR AVGDVLRDRF |
| 251 | DGASRVLYGN RGSNRASRAE LLRLEPEDPA HKPPSPHDLV YFEKSPNFCT |
| 301 | YSGRLGTAGT AGRACNSSSP ALDGCELLCC GRGHRTRTQR VTERCNCTFH |
| 351 | WCCHVSCRNC THTRVLHECL |

| | | | | | | |
|---|---|---|---|---|---|---|
| Start-End | Observed | Mr(expt) | Mr(calc) | Delta | Miss | Sequence |

| | | | | | | |
|---|---|---|---|---|---|---|
| 74-94 | 2180.05 | 2179.04 | 2179.12 | −0.08 | 1 | QNPGILHSVSGGLQSAVRECK |
| 102-115 | 1581.73 | 1580.72 | 1580.76 | −0.03 | 0 | WNCPTAPGPHLFGK |
| | | | | | | Carbamidomethyl (C) |
| 159-178 | 2129.99 | 2128.98 | 2128.88 | 0.10 | 0 | GPGGPDWHWGGCSDNIDFGR |
| 196-215 | 2399.17 | 2398.16 | 2398.12 | 0.05 | 1 | FLMNLHNNEAGRTTVFSEMR |
| | | | | | | 2 Oxidation (M) |
| 295-313 | 1915.92 | 1914.91 | 1914.90 | 0.01 | 1 | SPNFCTYSGRLGTAGTAGR |
| 359-370 | 1425.62 | 1424.61 | 1424.67 | −0.06 | 1 | NCTHTRVLHECL |

Notes:
*Match to: NCBI/SWISS PROT Accession No. P04628.
Score: 45
Expect: 1.4
Wnt-1 proto-oncogene protein precursor
Sequence Coverage: 29%

In addition, it can be seen from FIG. 12 that the expression level of Wnt-1 as enhanced by at least two-fold in the tumor portion than in the non-tumor portion amongst 6 of the additional eight paired-HCC specimens (Patient Nos. 10-17).

Based on the obtained results, in particular those shown in Table 2 and FIG. 12, Wnt-1 protein may be used as a biomarker for either detecting the predisposition of HCC in a subject or for predicting the prognosis of a subject having HCC.

Specifically, according to the results collected from patients studied so far, the prognosis of a subject having or suspected to have hepatocellular carcinoma may be evaluated based on a value of ratio obtained from comparing the level of Wnt-1 expression in the tumor portion with that in the non-tumor portion of a paired liver specimen taken from said subject, in which the subject will be evaluated to have:
(i) a prognosis of no more than 6 months if the obtained value of ratio is greater than 2;
(ii) a prognosis of 6 to 18 months if the obtained value of ratio is between 1 and 2; or
(iii) a prognosis of at least 18 months if the obtained value of ratio is less than 1.
   Discussion

The study of this invention analyzed NF-κB-associated signaling protein complexes in HCC tumor and non-tumor tissues by functional proteomic approach. The expression levels of Wnt-1 protein were incidentally found to be higher in tumor portions than in non-tumor portions of paired liver specimens taken from patients infected with at least one of HBV and HCV.

For the first time, we demonstrated that the level of Wnt-1 expression was enhanced in tumor portions than in non-tumor portions of paired liver specimens taken from patients infected with at least one of HBV and HCV. This enhancement is clinically related to the hepatocarcinogenesis of HCC. Most importantly, the overexpression of Wnt-1 protein is associated with NF-κB signaling. This is consistent with the data reported by Pikarsky et al. that NF-κB functions as a tumor promoter in inflammation-associated cancer (E. Pikarsky et al. (2004), Nature, 431:461-466).

Common manifestations of HBV and HCV infections include common histopathological changes in the liver, common clinical evolution from chronic hepatitis, cirrhosis and ultimately to HCC (Bréchot, C. 2001. In: Arias, I. M. editor-in-chief. The Liver: Biology and Pathobiology. 4th ed. Philadelphia (Pa.): Lippincott: P. 801-830). Clinically, although multimodality treatment protocols have been applied to treat HCC patients, the prognosis of this cancer is still very poor (A. Sangiovanni et al. (2004), Gastroenterology, 126:1005-1014; S. Ueno et al. (2001), Hepatology, 34:529-534; T. W. T. Leung et al. (2002), Cancer, 94:1760-1769; J. Bruix and J. M. Llovert (2002), Hepatology, 35:519-524).

The link of proto-oncogenic protein Wnt-1 with NF-κB activity was first reported by Boumat et al. in J. Neurosci. Res., 61:21-32, 2000. Their study showed that the Wnt-1-mediated survival of PC12 cells, a rat pheochromocytoma cell line of neural crest lineage, is dependent on NF-κB activation, and that stable expression of Wnt-1 increases NF-κB activity. The association of Wnt signaling with NF-κB pathway was noted earlier in studying the ubiquitin-dependent proteolysis by the proteasome. The key mediator of that pathway is β-catenin (K. Willer and R. Nusse (1998), Curr. Opin. Genet. Dev., 8; 95-102.). Moreover, a link of HBV with Wnt signaling has been reported recently in hepatoma cells that X-protein of HBV (HBx) may enhance stabilization of β-catenin, and is essential for the activation Wnt/β-catenin signaling (M. Y. Cha et al. (2004), Hepatology, 39:1683-1693).

There has yet to be reported the association of HCV infection with Wnt/β-catenin signaling. However, the heat-shock protein 27 (HSP27) has been identified by proteomic approach to interact with nonstructural protein 5A (NS5A) of HCV (Y. W. Choi et al. (2004), Biochem. Biophys. Res. Commun., 318: 514-519). Likewise, HSP70 showed a tendency toward overexpression in HCV-related HCC tumor tissues (M. Takashima et al. (2003), Proteomics, 3: 2487-2493). The induced heat shock proteins include some that help stabilize and repair partly denatured cell proteins, and are closely related to ubiquitin-dependent proteolysis pathway (H. Shimura et al. (2004), J. Biol. Chem., 279: 4869-4876), and are also linked to NF-κB signaling and cell survival (R. Ran et al. (2004), Genes Dev., 18:1466-1481). Thus, only an indirect association of HCV infection with Wnt/β-catenin signaling can now be linked together. This speculation needs further investigation.

Accumulating evidence shows that activation of NF-κB inhibits apoptosis, and that inhibition of NF-κB enhances antitumor therapy through increased apoptosis (A. A. Berg, and D. Baltimore (1996), Science, 274:782-784; D. J. Van Antwerp et al. (1996), Science, 274:787-789; C. Y. Wang et al. (1999), Nat. Med., 5:421-427). It was also reported that activation of IEX-1L gene, an apoptosis inhibitor, and the induction of inhibitor of apoptosis proteins (c-IAP1 and c-IAP2) may be involved in NF-κB-mediated cell survival (M. X. Wu et al. (1998), Science, 281: 998-1001; C. Y. Wang et al. (1998), Science, 281:680-1683). Whether NF-κ-associated Wnt-1 protein expression in HBV- and HCV-infected HCC tumor and nontumor tissues is related to these anti-apoptosis factors remains to be elucidated.

Accumulating evidence suggests that the evolutionarily conserved Wnt-signaling pathway may have pivotal roles during the development of many organs (K. M. Cardigan and R. Nusse (1997), Genes Dev., 11:3286-3305; A. Ruiz i Altaba et al. (2002), Nature Rev. Cancer, 2:361-370; F. J. T. Staal et al. (2005), Nature Rev. Immunol, 5: 21-30), and dysregulated Wnt-signaling is a key factor in the initiation of various tumors and the development of diseases (P. Polakis, (2000), Genes Dev., 14:1837-1641; J. Taipale and P. A. Beachy (2001), Nature, 411: 349-354; D. Kalderon (2002), Trends Cell. Biol., 12: 523-531; A. Ruiz i Altaba et al. (2002), Nature Rev. Cancer, 2:361-370; J. R. Miller (1999), Oncogene, 18:7860-7872; W. J. Nelson and R. Nusse (2004), Science, 303: 1483-1487).

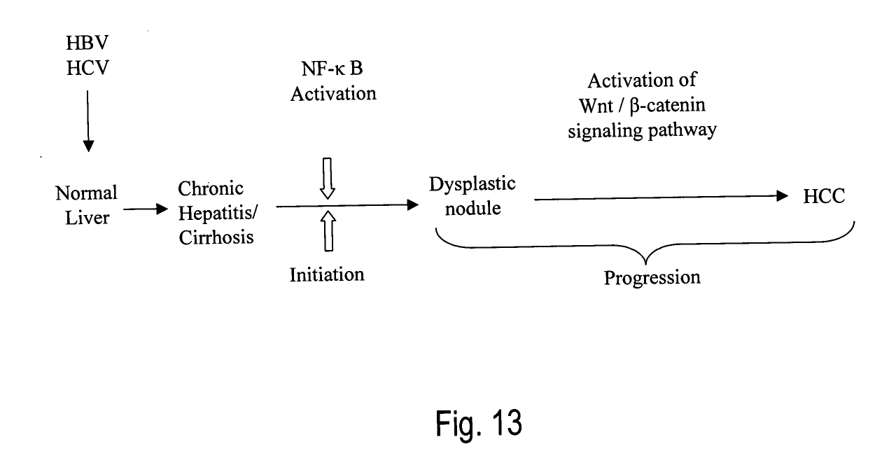

Our finding that the expression of Wnt-1 protein is enhanced in both HBV- and/or HCV-related HCC tumor tissues than in non-tumor tissues is relevant to cancer formation. It is proposed that NF-κB-associated Wnt-1 protein may serve as a common denominator of HBV- and HCV-related hepatocarcinogenesis. The scenario of this proposal in terms of the enhanced expression of NF-κB-associated Wnt-1 protein is shown in FIG. 13. A possible scientific basis for this proposal is that the Wnt proteins (Wnts) are ligands for the Frizzled (Fz) receptors, which resemble typical G protein-coupled receptors (K. M. Cardigan and R. Nusse (1997), Genes Dev., 11:3286-3305; J. R. Miller et al (1999), Oncogene, 18:7860-7872). Consequently, although Wnt proteins act extracellularly on membrane Fz receptors, Wnt-signaling may be involved in the regulation of a multiprotein complex including NF-κB intracellularly by the mechanism of receptor-mediated endocytosis. It is presumed that NF-κB activated by HBV and HCV infections may further interact with Wnts and other regulatory factors to control cell growth. Questions about how the NF-κB signaling enhances Wnt-1 protein expression and how these complexes associate with Wnt-1 proteins in HBV- and HCV-related hepatocarcinogenesis still need to be clarified. Further studies are required to explore the connections amongst HBV and/or HCV, NF-κB, Wnt-1, β-catenin pathway and HCC in more complex pathophysiological contexts.

It has been suggested that the Wnt signaling pathway might be used as a therapeutic target for designing new treatment regimens in children with medulloblastoma (R. J. Gilbertson (2004), Lancet Oncol., 5:209-218) and in people with head and neck squamous cell carcinomas (C. S. Rhee (2002), Oncogene, 21:6598-6605). Theoretically, it is feasible by targeting Wnt signaling pathway (J. Taipale and P. A. Beachy (2001), Nature, 411.349-354; Kalderon, D. (2002), Trends Cell. Biol., 12:523-531; A. Ruiz i Altaba et al. (2002), Nature Rev. Cancer, 2:361-370; R. J. Gilbertson (2004), Lancet Oncol., 5:209-218; C. S. Rhee et al. (2002), Oncogene, 21:6598-6605) together with NF-κB signaling (A. Lin and M. Karin (2003), Semin. Cancer Biol., 13:107-114; C. Y. Wang et al. (1999), Nat. Med., 5:421-427) for designing highly effective therapeutic agents in the treatment of HCC and for chemoprevention of hepatocarcinogenesis (W. K. Hong and M. B. Sporn (1997), Science, 278:1073-1077; W. J. Nelson and R. Nusse (2004), Science, 303:1483-1487).

In conclusion, our study suggests that enhanced expression of NF-κB associated Wnt-1 protein may constitute a common mechanism of HBV- and HCV-related hepatocarcinogenesis.

All patents and literature references cited in the present specification are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

We claim:

1. A method for determining predisposition to hepatocellular carcinoma in a subject having or suspected to have a hepatic nodule, comprising:
separating a liver specimen taken from the subject into a tumor portion and a non-tumor portion;
detecting the levels of Wnt-1 expression in the tumor portion and the non-tumor portion, respectively;
comparing the detected level of Wnt-1 expression in the tumor portion with that in the non-tumor portion to obtain a value of ratio; and
determining whether or not the subject is predisposed to hepatocellular carcinoma based on the obtained value of ratio, wherein the subject is determined to be predisposed to hepatocellular carcinoma if the obtained value of ratio is greater than 1.

2. The method according to claim 1, wherein the subject has been infected with at least one of hepatitis B virus and hepatitis C virus.

3. The method according to claim 1, wherein the liver specimen is taken from a part of the subject's liver containing a hepatic nodule.

4. The method according to claim 1, wherein the liver specimen is taken from the subject via a surgical operation selected from segmentectomy and right lobectomy.

5. The method according to claim 1, wherein the liver specimen is taken from the subject via liver biopsy, aspiration or peritoneoscopy.

6. The method according to claim 1, wherein an increase in the detected level of Wnt-1 expression in the tumor portion as compared to that in the non-tumor portion is associated with NF-κB activation.

7. The method according to claim 1, wherein the levels of Wnt-1 expression are detected by quantifying mRNA transcript of Wnt-1 gene.

8. The method according to claim 7, wherein quantifying mRNA transcript of Wnt-1 gene is conducted using at least one of the following methodologies: hybridization, Northern blotting, and quantitative polymerase chain reaction.

9. The method according to claim 1, wherein the levels of Wnt-1 expression are detected by quantifying Wnt-1 protein.

10. The method according to claim 9, wherein quantifying Wnt-1 protein is conducted using at least one of the following methodologies: gel electrophoresis, Western blotting, enzyme immunoassay such as enzyme linked immunosorbent assay, radioimmunoassay, immunohistochemistry, and proteomics.

11. The method according to claim 9, wherein quantifying Wnt-1 protein is conducted using an antibody-based binding moiety which specifically binds Wnt-1 protein.

12. The method according to claim 11, wherein the antibody-based binding moiety is labeled with a detectable label.

13. The method according to claim 13, wherein the label is selected from the group consisting of a radioactive label, a hapten label, a fluorescent label, and an enzymatic label.

14. The method according to claim 11, wherein the antibody-based binding moiety is an antibody.

15. A method for evaluating the prognosis of a subject having or suspected to have hepatocellular carcinoma, comprising:
separating a liver specimen taken from the subject into a tumor portion and a non-tumor portion;
detecting the levels of Wnt-1 expression in the tumor portion and the non-tumor portion, respectively;
comparing the detected level of Wnt-1 expression in the tumor portion with that in the non-tumor portion to obtain a value of ratio; and
evaluating the prognosis of the subject based on the obtained value of ratio, wherein the subject is evaluated to have:
(i) a prognosis of no more than 6 months if the obtained value of ratio is greater than 2;
(ii) a prognosis of 6 to 18 months if the obtained value of ratio is between 1 and 2; or
(iii) a prognosis of at least 18 months if the obtained value of ratio is less than 1.

16. The method according to claim 15, wherein the subject has been infected with at least one of hepatitis B virus and hepatitis C virus.

17. The method according to claim 15, wherein the liver specimen is taken from a part of the subject's liver containing a hepatic nodule.

18. The method according to claim 15, wherein the liver specimen is taken from the subject via a surgical operation selected from segmentectomy and right lobectomy.

19. The method according to claim 15, wherein the liver specimen is taken from the subject via liver biopsy, aspiration or peritoneoscopy.

20. The method according to claim 15, wherein an increase in the detected level of Wnt-1 expression in the tumor portion as compared to that in the non-tumor portion is associated with NF-κB activation.

21. The method according to claim 15, wherein the levels of Wnt-1 expression are detected by quantifying mRNA transcript of Wnt-1 gene.

22. The method according to claim 21, wherein quantifying mRNA transcript of Wnt-1 gene is conducted using at least one of the following methodologies: hybridization, Northern blotting, and quantitative polymerase chain reaction.

23. The method according to claim 15, wherein the levels of Wnt-1 expression are detected by quantifying Wnt-1 protein.

24. The method according to claim 23, wherein quantifying Wnt-1 protein is conducted using at least one of the following methodologies: gel electrophoresis, Western blotting, enzyme immunoassay such as enzyme linked immunosorbent assay, radioimmunoassay, immunohistochemistry, and proteomics.

25. The method according to claim 23, wherein quantifying Wnt-1 protein is conducted using an antibody-based binding moiety which specifically binds Wnt-1 protein.

26. The method according to claim 25, wherein the antibody-based binding moiety is labeled with a detectable label.

27. The method according to claim 26, wherein the label is selected from the group consisting of a radioactive label, a hapten label, a fluorescent label, and an enzymatic label.

28. The method according to claim 25, wherein the antibody-based binding moiety is an antibody.
